(19) United States
(12) Patent Application Publication
(10) Pub. No.: US 2026/0364071 A1
(43) Pub. Date: Nov. 19, 2026

(54) SOLID DISPERSION OF MYELOID KINOME INHIBITORS AND PHARMACEUTICAL COMPOSITION COMPRISING THE SAME

(71) Applicant: Hanmi Pharm. Co., Ltd., Hwaseong-si, Gyeonggi-do (KR)

(72) Inventors: Kwangyoung LEE, Hwaseong-Si (KR); Taekkwan KWON, Hwaseong-Si (KR); Yongil KIM, Hwaseong-Si (KR)

(21) Appl. No.: 19/487,409

(22) PCT Filed: May 31, 2024
(86) PCT No.: PCT/KR2024/007424

§ 371 Date: Nov. 24, 2025

Related U.S. Application Data
(60) Provisional application No. 63/505,318, filed on May 31, 2023.

(51) Int. Cl. A61K 31/506; A61K 9/20; A61K 9/28

(52) U.S. Cl. CPC A61K 31/506; A61K 9/2009; A61K 9/2013; A61K 9/2018; A61K 9/2027; A61K 9/2054; A61K 9/284

(57) ABSTRACT

A solid dispersion of 5-chloro-N-(3-cyclopropyl-5-{[(3R,5S)-dimethylpiperazin-1-yl]methyl}phenyl)-4-(6-methyl-1H-indol-3-yl)pyrimidin-2-amine and processes for preparing the solid dispersion are provided herein. Also, a pharmaceutical composition comprising a solid dispersion of 5-chloro-N-(3-cyclopropyl-5-{[(3R,5S)-dimethylpiperazin-1-yl]methyl}phenyl)-4-(6-methyl-1H-indol-3-yl)pyrimidin-2-amine and uses thereof are provided herein.

### TECHNICAL FIELD

A solid dispersion of 5-chloro-N-(3-cyclopropyl-5-{[(3R,5S)-dimethylpiperazin-1-yl]methyl}phenyl)-4-(6-methyl-1H-indol-3-yl)pyrimidin-2-amine is provided herein. Also, a pharmaceutical composition comprising a solid dispersion of 5-chloro-N-(3-cyclopropyl-5-{[(3R,5S)-dimethylpiperazin-1-yl]methyl}phenyl)-4-(6-methyl-1H-indol-3-yl)pyrimidin-2-amine is provided herein.

### BACKGROUND ART

FMS-like tyrosine kinase 3 (FLT3), a receptor tyrosine kinase, is normally expressed in hematopoietic progenitor cells and plays an important role in the expression of normal stem cells and the immune system. Abnormal overexpression and mutations of FLT3 are often found in patients with leukemia. In particular, various mutations of FLT3, such as D835V, D835Y and internal tandem duplication (ITD), are found in acute myeloid leukemia (AML). AML is a hematopoietic stem cell disorder characterized by abnormal proliferation and differentiation of blast cells in myeloid and peripheral blood. FLT3 has recently been regarded as one of the most important targets in the therapeutic aspects of AML.

Likewise, the SYK and JAK1/2 intracellular kinases as well as c-KIT alternative receptor kinase, mediate oncogenic signaling in AML that can promote drug resistance to certain FLT3 inhibitors.

Spleen tyrosine kinase (SYK), a non-receptor protein tyrosine kinase, is an important mediator of immune receptor signaling in B cells, mast cells, neutrophils, and macrophages. Not only SYK is expressed in 90% of AML blasts, also the activity of SYK were related with an unfavorable outcome independent of age, cytogenetics, and leukocyte count in AML patients. Typically, phosphorylation of spleen tyrosine kinase (SYK), which is frequently overexpressed and constitutively activated in AML, is increased in cells stimulated via chemokine receptors and integrins. Activation of SYK could induce the leukemia protection effect in the bone marrow microenvironment. In addition, cytokines released by bone marrow stromal cells could protect leukemia cells from the cytotoxic effects of drugs through induction of JAK/STAT signaling (Blood. 2017, 130(6), 789-802, Blood Adv. 2019, 3(7), 1061-1072).

The Janus Associated Kinase-Signal Transducers and Activators of Transcription (JAK-STAT) signaling pathway plays a pivotal role in hematopoietic growth factor signaling. Hyperactive JAK-STAT signaling is implicated in the pathogenesis of myeloid malignancies, including acute myeloid leukemia (AML). The significant headway in understanding the biology of AML has led to an explosion of novel therapeutics with mechanistic rationale for the treatment of newly diagnosed and relapsed/refractory (R/R) AML. Selective targeting of the JAK-STAT pathway has proven to be an effective therapeutic strategy in myeloproliferative neoplasms and is also being evaluated in related myeloid malignancies, including AML. (Blood Reviews, 2020, 40, 100634).

c-KIT mutations are reported in nearly all systemic mastocytosis, 20% to 40% core-binding factor (CBF) acute myeloid leukemia (AML), and approximately 20% high-grade myelodysplastic syndrome (MDS) and MDS-derived AML. c-KIT mutation in AML confers increased risk of relapse and decreased overall survival. (Br J Haematol. 2003, 121(5), 775-777, Leuk Res. 2006, 30(10), 1235-1239, J Clin Oncol. 2006, 24(24), 3904-3911).

5-chloro-N-(3-cyclopropyl-5-{[(3R,5S)-dimethylpiperazin-1-yl]methyl}phenyl)-4-(6-methyl-1H-indol-3-yl)pyrimidin-2-amine is a potent inhibitor of Myeloid Kinome (FLT3, SYK, JAK and mutant c-KIT kinases) and can be useful for treating B-lineage neoplasms, such as acute myeloid leukemia. The compound and its synthesis have been described in PCT application publication WO2020/022600A1, which is incorporated by reference in its entirety herein. There remains interest in developing efficacious and safe formulations for this and other related biologically active molecules.

### DISCLOSURE OF INVENTION

### Technical Problem

A solid dispersion of 5-chloro-N-(3-cyclopropyl-5-{[(3R,5S)-dimethylpiperazin-1-yl]methyl}phenyl)-4-(6-methyl-1H-indol-3-yl)pyrimidin-2-amine is provided herein. Also, a pharmaceutical composition comprising a solid dispersion of 5-chloro-N-(3-cyclopropyl-5-{[(3R,5S)-dimethylpiperazin-1-yl]methyl}phenyl)-4-(6-methyl-1H-indol-3-yl)pyrimidin-2-amine is provided herein.

### Solution to Problem

Described herein are pharmaceutical formulations and compositions comprising the compound 5-chloro-N-(3-cyclopropyl-5-{[(3R,5S)-dimethylpiperazin-1-yl]methyl}phenyl)-4-(6-methyl-1H-indol-3-yl)pyrimidin-2-amine as well as amorphous forms of the same and methods of uses thereof. 5-chloro-N-(3-cyclopropyl-5-{[(3R,5S)-dimethylpiperazin-1-yl]methyl}phenyl)-4-(6-methyl-1H-indol-3-yl)pyrimidin-2-amine is

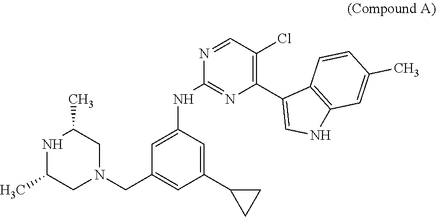

given in Compound A or a pharmaceutically acceptable salt, solvate, stereoisomer or prodrug thereof.

The present invention includes amorphous forms of Compound A, pharmaceutical compositions comprising Compound A, and methods of treating cancer by administering Compound A or compositions comprising Compound A. Relevant synthesis of Compound A and its method of use are disclosed in PCT application publications WO2020022600, WO2020171646, WO2020171649, WO2020262974, WO2021066443, which are incorporated by reference in their entirety herein. Thus, in one aspect, the present invention provides solid dispersion comprising Compound A or a pharmaceutically acceptable salt, solvate, stereoisomer or prodrug thereof, and a polymeric carrier. In a specific embodiment Compound A is in a solid dispersion and is substantially amorphous and dispersed in a polymer matrix of the polymeric carrier in an amount of from about 10 parts to about 55 parts by weight based on 100 parts by weight of Compound A.

In an embodiment, the present invention provides solid dispersion comprising Compound A in which the solid dispersion is formed by solvent evaporation methods such as spray-drying and fluid bed granulation.

In yet another embodiment, the polymeric carrier is a hydrophilic carrier. In a more specific embodiment, the polymeric carrier is hydroxypropyl methyl cellulose. In another more specific embodiment, the polymeric carrier is hydroxypropyl methyl cellulose acetate succinate.

In another aspect, the present invention provides a pharmaceutical composition comprising Compound A solid dispersion and pharmaceutically acceptable excipients. The pharmaceutical composition comprises a) from about 20% to about 50% w/w of Compound A solid dispersion, b) from about 25% to about 70% w/w of filler/binder, c) from about 4.0% to about 20% w/w of disintegrant, d) from about 0.25% to about 5.0% w/w of glidant/adsorbent and e) from about 0.5% to about 2.5% w/w of lubricant, in which the Compound A solid dispersion comprises substantially amorphous Compound A dispersed in a polymer matrix of a hydrophilic polymeric carrier in an amount of from about 64.5% to about 91% w/w Compound A based on the combined weight of the Compound A and the hydrophilic polymeric carrier.

In an embodiment, the pharmaceutical composition is a solid oral dosage form selected from the group consisting of tablets, powders, granules, caplets and pills. In a specific embodiment, the solid oral dosage form is a tablet.

In another embodiment, the Compound A solid dispersion of the pharmaceutical composition is formed by spray-drying.

In certain embodiments, the formulation or solid oral dosage form according to the pharmaceutical composition provides improved pharmacokinetics (PK) properties as compared to the reference formulation comprising the same amount of Compound A. In one embodiment, the PK properties include drug exposure (Cₘₐₓ), and area under the curve (AUC). In a specific embodiment, the pharmaceutical composition is a tablet in which the tablet provides about 2-fold to about 3-fold increase in drug exposure, e.g., maximal plasma concentration (Cₘₐₓ) of Compound A, over a reference tablet comprising an identical amount of Compound A but not comprising an amorphous solid dispersion of Compound A. In another specific embodiment, the pharmaceutical composition is a tablet in which the tablet provides about 2-fold to about 3-fold increase in area under the curve (AUC) over a reference tablet comprising an identical amount of Compound A but not comprising an amorphous solid dispersion of Compound A. In a further specific embodiment, the drug exposure or the area under the curve is measured in beagle dogs.

In yet another aspect, the present invention provides a pharmaceutical composition for capsule comprising Compound A solid dispersion and pharmaceutically acceptable excipients. The pharmaceutical composition comprises: a) from about 50% to about 97.5% w/w of Compound A solid dispersion, b) from about 0% to about 48% w/w of filler/binder, c) from about 0% to about 20% w/w of disintegrant, d) from about 0% to about 4.0% w/w of glidant/adsorbent and e) from about 0.2% to about 4.0% w/w of lubricant, in which the Compound A solid dispersion comprises substantially amorphous Compound A dispersed in a polymer matrix of a hydrophilic polymeric carrier in an amount of from about 64.5% to about 91% w/w Compound A based on the combined weight of the Compound A and the hydrophilic polymeric carrier.

In an aspect further yet, the present invention provides solid oral formulation comprising from about 20% to about 50% w/w of Compound A solid dispersion, wherein this Compound A solid dispersion comprises substantially amorphous Compound A dispersed in a polymer matrix of hydroxypropyl methyl cellulose in an amount of from about 64.5% to about 91% w/w Compound A based on the combined weight of the Compound A and hydroxypropyl methyl cellulose, such that this solid oral formulation provides, over a reference tablet comprising an identical amount of Compound A but not comprising an amorphous solid dispersion of Compound A, about 2-fold to about 3-fold increase in drug exposure or area under the curve (AUC).

### Advantageous Effects of Invention

In still another aspect, the present invention provides methods to treat diseases or conditions using the pharmaceutical composition of the present invention. Exemplary diseases or conditions include B-lineage neoplasms such as leukemia. In a specific embodiment, the disease or condition is acute myeloid leukemia.

### BRIEF DESCRIPTION OF DRAWINGS

FIGS. 1A to 1E show scanning electron microscopy photographs of solid dispersions of Compound A for Examples 1-3 (FIGS. 1A-1C, respectively) and Comparative Examples 1-3 (FIGS. 1D-1F, respectively);

FIG. 2 is a graph showing saturation solubility for Compound A solid dispersions of Examples 1-3 and Comparative Example 1;

FIG. 3A and FIG. 3B are respectively the X-ray powder diffraction (XRPD) pattern and differential scanning calorimetry (DSC) curve for a Compound A solid dispersion of Example 1, and FIG. 3C and FIG. 3D are the XRPD pattern and DSC curve for Comparative Example 1 formulation;

FIGS. 4A to 4D are XRPD patterns of a Compound A solid dispersion tablet under different storage conditions and FIG. 4E is an XRPD pattern for a crystalline Compound A monohydrate of Comparative Example 1;

FIG. 5 is a graph comparing the mean plasma concentration-time profiles for Example C and Comparative Example B tablets.

### MODE FOR THE INVENTION

### Certain Terminology

Reference will now be made in detail to certain embodiments, examples of which are illustrated herein. While enumerated embodiments will be described, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in this disclosure including, but not limited to, patents, patent applications, articles, books, manuals, and treatises are hereby expressly incorporated by reference in their entirety for any purpose.

The term “about” when used before a numerical value indicates that the value may vary within a reasonable range, such as within ±10%, ±5% or ±1% of the stated value. Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

As used herein, the term “comprising” is intended to mean that the compositions and methods, etc., include the recited elements, but do not exclude others. “Consisting essentially of” when used to define compositions and methods, shall mean excluding other elements of any essential significance to the combination for the intended use, but not excluding elements that do not materially affect the characteristic(s) of the compositions or methods. “Consisting of” shall mean excluding elements not specifically recited. Embodiments defined by each of these transition terms are within the scope of this invention.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. In certain embodiments, a “therapeutically-effective amount” of Compound A refers to such dosages and/or administration for such periods of time necessary to inhibit Myeloid Kinome(FLT3, SYK, JAK and mutant c-KIT kinases), such as wild-type or mutants. Moreover, a therapeutically effective amount may be one in which the overall therapeutically-beneficial effects outweigh the toxic or undesirable side effects. A therapeutically-effective amount of Compound A may vary according to disease state, age and weight of the subject being treated. Thus, dosage regimens are typically adjusted to the individual requirements in each particular case and are within the skill in the art. In certain embodiments, an appropriate daily dose for administration of Compound A to an adult human may be from about 10 mg to about 2500 mg; or from about 50 mg to about 1000 mg, although the upper and lower limits may be exceeded when indicated. A daily dosage of Compound A can be administered as a single dose, in divided doses, or, for parenteral administration, it may be given as subcutaneous injection.

“Bioavailability” refers to the percentage of Compound A dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which Compound A is absorbed into the general circulation when the pharmaceutical composition is taken orally as compared to intravenous injection.

As used herein, the term “area under the curve,” or its acronym “AUC” is a pharmacokinetic term used to refer to a method of measurement of bioavailability or extent of absorption of a drug based on a plot of an individual (e.g., a human or another test animal) or pool of individual's blood plasma concentrations sampled at frequent intervals. The AUC means the area under the administered drug's (e.g. Compound A) plasma concentration-time curve, as calculated by the trapezoidal rule over the complete sample collection interval. The AUC is a measure of total bioavailability and is directly proportional to the total amount of unaltered drug in that individual or test animal's blood plasma. AUC is typically given for the time interval zero to infinity, however, clearly plasma drug concentrations cannot be measured “to infinity” for a patient so a mathematical equation is used to estimate the AUC from a limited number of concentration measurements.

As used herein, the term “AUC_{0-∞}” refers to the area under the plasma concentration-time curve extrapolated to infinity.

As used herein, the term “maximal plasma concentration” or its symbol “Cₘₐₓ” is the highest plasma concentration of the drug or delivery agent observed within the sampling interval. It is a measure of drug exposure.

As used herein, the symbol “Tₘₐₓ” denotes the time post-dose at which maximal plasma concentration of the drug is observed.

As used herein, the term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point). The absence of crystalline drug in an amorphous dispersion may be characterized by modulated differential scanning calorimetry (mDSC), X-ray powder diffraction (XRPD), near infrared spectroscopy (NIR), or any other standard analytical technique. For example, mDSC assesses the thermal properties of an amorphous dispersion. Analysis by mDSC will yield a single glass transition temperature. mDSC can also detect crystalline phase separation, as the crystalline phase will show a unique thermal signal. XRPD uses X-rays to identify crystal form in solid powders and can be used, for example to confirm a solid dispersion is a single amorphous phase, with no measurable crystalline material.

As used herein, the term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

As used herein, the term “substantially amorphous” as used herein is intended to mean that greater than 60%; or greater than 65%; or greater than 70%; or greater than 75%; or greater than 80%; or greater than 85%; or greater than 90%; or greater than 95%; or greater than 99% of the compound present in a composition is in amorphous form. “Substantially amorphous” can also refer to material which has no more than about 20% crystallinity, or no more than about 10% crystallinity; or no more than about 5% crystallinity; or no more than about 2% crystallinity; or no more than about 1% crystallinity.

As used herein, the term “solid dispersion” means a system in a solid state comprising at least two components, wherein one component (a drug, e.g. Compound A) is dispersed throughout the other component, the polymeric carrier. In a solid dispersion, the component of polymeric carrier forms a matrix and a molecular dispersion of the drug takes place within this polymer matrix. In addition to the two components, a solid dispersion may also include in a minor amount excipients such as glidant/adsorbent. The compound (either crystalline or amorphous particles, optionally micronized or nanoparticles) is uniformly dispersed within the polymer matrix. In some embodiments, a solid dispersion includes Compound A molecularly dispersed with a polymer. Preferably the solid dispersion exists as a one phase system.

As used herein, the term “molecularly dispersed” refers to the random distribution of a compound (e.g. Compound A) with a polymer. In certain embodiments, the compound is present in the polymer in a final state of subdivision. See, e.g., M. G. Vachon et al., J Microencapsulation 14:281-301 (1997) and Vandelli et al., J Microencapsulation, 10: 55-65 (1993). In some embodiments, a compound (e.g. Compound A) may be dispersed within a matrix formed by the polymer in its solid state such that the compound is immobilized in its amorphous form. Whether a compound is molecularly dispersed in a polymer may be evidenced in a variety of ways, e.g., by the resulting solid molecular complex having a single glass transition temperature, that is intermediate in value between that of the drug and that of the polymer carrier.

As used herein, the term “amorphous solid dispersion” refers to a solid dispersion comprising a drug and a polymeric carrier, wherein the drug is non-crystalline and substantially amorphous. An amorphous solid dispersion of the drug can be prepared by various manufacturing processes such as, but not limited to, solvent evaporation (also known as solvent processing), spray-drying, co-precipitation, or hot melt extrusion. A spray-dried dispersion (SDD) is a single-phase, amorphous molecular dispersion of a drug in a polymer matrix; it is an amorphous solid in which the drug is molecularly “dissolved” in a solid matrix. A spray-dried dispersion can be made by dissolving the drug and a polymer in an organic solvent to produce a solution, followed by evaporating, e.g., spray-drying the solution. Techniques for preparing solid dispersions of an amorphous drug in a polymer are disclosed in, for example, U.S. Pat. No. 9,095,585 the contents of which are hereby incorporated by reference in their entirety herein.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

A “prodrug” refers to a derivative of a compound of the present disclosure that will be converted to the compound in vivo.

As used herein, the term “formulation” or “pharmaceutical formulation” refers to a pharmaceutical composition comprising at least one therapeutically active agent and a pharmaceutically acceptable excipient or carrier. A non-limiting example of formulations includes tablets, capsules, gel capsules, syrup, liquid, gel, suspension, solid dispersion, or combinations thereof.

As used herein, the term “dosage form” refers to one or more pharmaceutical compositions which provides a specific amount of a therapeutically active agent, such as a unit dose. In one embodiment, a dosage form can be provided in one or more pharmaceutical compositions. For example, if a subject is to be administered with 200 mg of a therapeutically active agent at a time (unit dose), a dosage form can comprise two tablets each containing 100 mg of the therapeutically active agent, wherein the two tablets are the same pharmaceutical composition.

As used herein, the term “filler/binder” generally refers to any pharmaceutically acceptable substance (such as any pharmaceutically acceptable film) that can be used to bind together the active and inert components of the carrier to maintain cohesive and discrete portions. Filler/binders may also serve to stabilize compounds. Examples of filler/binders may include starch, saccharides, disaccharides, sucrose, lactose, lactose monohydrate, polysaccharides, cellulose, cellulose ethers, hydroxypropylcellulose, sugar alcohols, xylitol, sorbitol, maltitol, microcrystalline cellulose, calcium or sodium carbonate, dicalcium phosphate, compressible sugars, dibasic calcium phosphate dehydrate, mannitol, and tribasic calcium phosphate.

As used herein, the term “disintegrant” generally refers to a substance which, upon addition to a solid preparation, facilitates its break-up or disintegration after administration and permits the release of an active ingredient as efficiently as possible to allow for its rapid dissolution. Examples of disintegrants may include maize starch, sodium starch glycolate, croscarmellose sodium, crospovidone, microcrystalline cellulose, modified corn starch, sodium carboxymethyl starch, povidone, pregelatinized starch, and alginic acid.

As used herein, the term “glidant/adsorbent” generally refers to substances used in tablet and capsule formulations to improve flow-properties during tablet compression, prevent unwanted sticking to the die and to produce an anti-caking effect. Examples of glidant/adsorbents may include colloidal silicon dioxide, talc, fumed silica, starch, starch derivatives, and bentonite.

The term “lubricant” generally refers to a substance that is added to a powder blend to prevent the compacted powder mass from sticking to the equipment during the tableting or encapsulation process. A lubricant can aid the ejection of the tablet form the dies, and can improve powder flow. Examples of lubricants may include magnesium stearate, stearic acid, silica, fats, calcium stearate, polyethylene glycol, sodium stearyl fumarate, or talc; and solubilizers such as fatty acids including lauric acid, oleic acid, and C8/C10 fatty acid.

As used herein, the notation “% w/w” refers to the weight content of a component of interest is calculated based on its ratio to the total weight of a composition comprising that component.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

As used herein, a “subject” can be a human, non-human primate, mammal, rat, mouse, cow, horse, pig, sheep, goat, dog, cat and the like.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

### Compound A Solid Dispersion

One aspect of the present invention is an amorphous from of Compound A

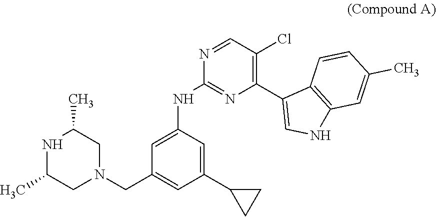

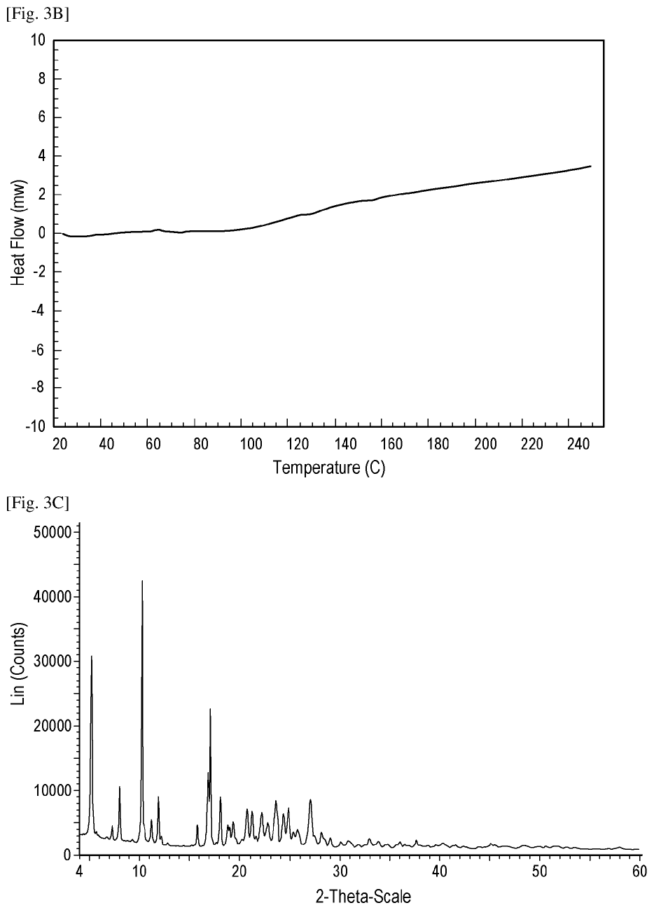

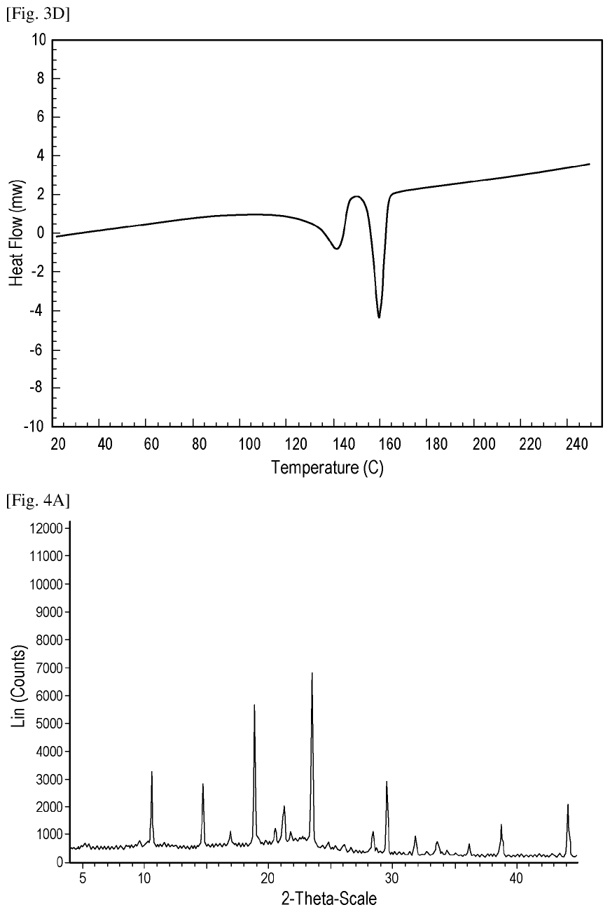

or a pharmaceutically acceptable salt, solvate, or solvate salt thereof. In another specific embodiment, the amorphous form exhibits an X-ray powder diffraction (XRPD) pattern substantially similar to the pattern in FIG. 3A. In another embodiment, the amorphous form of Compound A exhibits an X-ray powder diffraction (XRPD) pattern as in FIG. 3A. In another embodiment, the amorphous form exhibits a differential scanning calorimetry (DSC) curve substantially similar to FIG. 3B. In another embodiment, the amorphous form exhibits a differential scanning calorimetry (DSC) curve as in FIG. 3B.

In another embodiment, the amorphous form of Compound A is from different storage conditions. In another embodiment, the amorphous is provided in a pharmaceutical composition. In another embodiment, the composition relates to a solid dispersion.

In one aspect, the present invention provides the solid dispersion of Compound A.

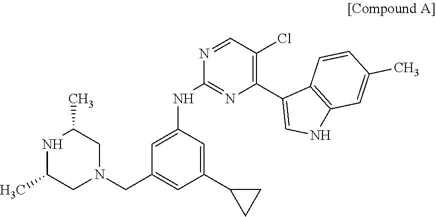

For the purposes of the solid dispersion mentioned above, forms of Compound A other than the compound represented by Compound A are contemplated as well as starting material in the manufacture of the solid dispersion. Other forms of Compound A suitable for solid dispersion starting material may include any hydrate of the Compound A, such as monohydrate, dehydrate, trihydrate; an anhydrate; any pharmaceutically acceptable salt of the Compound A such as hydrochloride; any solvate, such as ethanolate; and any hydrate of a pharmaceutically acceptable salt of the Compound A. These other forms can be employed as starting material for preparing solid dispersion by using solvent evaporation methods such as spray-drying or fluid bed granulation.

The present disclosure thus relates to pharmaceutical compositions and formulations comprising Compound A, wherein the composition is a solid dispersion. In one embodiment, the solid dispersion is formed by solvent evaporation (also known as solvent processing), hot-melt extrusion or spray drying methods. In one embodiment, the solid dispersion is a spray-dried dispersion (SDD).

In one embodiment, the solid dispersion is prepared by solvent evaporation where after the therapeutically active agent and the polymeric carrier are both dissolved in the same solvent or a solvent mixture, the solvent or the solvent mixture is rapidly removed by evaporation or by mixing with a non-solvent. Rapid removal of the solvent or the solvent mixture can be achieved using spray-drying, spray-coating (pan-coating, fluidized bed coating, etc.), and precipitation by rapid mixing of the polymer and drug solution with CO₂, water, or other non-solvent. In one embodiment, the solid dispersion is a supersaturated solid solution where the concentration of the therapeutically active agent in the polymeric carrier is above its equilibrium value.

In one embodiment, the solid dispersion has a single glass transition temperature. A single glass transition temperature for a solid dispersion indicates a high degree of homogeneity.

In one embodiment, the solid dispersion comprises one or more water-soluble polymer. In one embodiment, the solid dispersion comprises one or more cellulose derivatives. In one embodiment, the solid dispersion comprises one or more water-soluble cellulosic polymer. In one embodiment, the solid dispersion comprises one or more cellulosic or non-cellulosic polymer. In one embodiment, the solid dispersion comprises one or more polymer that are neutral or ionizable in aqueous solution. In one embodiment, the solid dispersion comprises one or more polymers that are ionizable and cellulosic. In one embodiment, the solid dispersion comprises one or more polymers that are ionizable cellulosic polymers. In one embodiment, the solid dispersion comprises one or more amphiphilic polymers. In one embodiment, the solid dispersion comprises one or more hydrophilic polymer. In one embodiment, the solid dispersion comprises one or more water-soluble hydrophilic polymer. In one embodiment of the pharmaceutical composition of the present disclosure, Compound A or a pharmaceutically acceptable salt, solvate, stereoisomer or prodrug thereof, is present in about 10% to about 90% by weight of the total composition (which can include one or more additional therapeutically active agents and/or other pharmaceutically acceptable excipients and/or coatings), including all values and subranges therebetween. In one embodiment of the solid dispersion, Compound A or a pharmaceutically acceptable salt, solvate, stereoisomer or prodrug thereof, is present in about 10%, about 15%, about 20%, about 25%, about 30%, about 35%, about 40%, about 45%, about 50%, about 55%, about 60%, about 65%, about 70%, about 75%, about 80%, about 85%, or about 90% by weight of the total composition, including all values therebetween. In one embodiment, Compound A or a pharmaceutically acceptable salt, solvate, stereoisomer or prodrug thereof, is present in about 10% to about 80%, about 10% to about 70%, about 10% to about 60%, about 10% to about 50%, about 10% to about 40%, about 10% to about 30%, about 10% to about 20%, about 15% to about 85%, about 15% to about 75%, about 15% to about 65%, about 15% to about 55%, about 15% to about 45%, about 15% to about 35%, about 15% to about 25%, about 20% to about 80%, about 20% to about 70%, about 20% to about 60%, about 20% to about 50%, about 20% to about 40%, about 20% to about 30%, about 25% to about 85%, about 25% to about 75%, about 25% to about 65%, about 25% to about 55%, about 25% to about 45%, about 25% to about 35%, about 30% to about 80%, about 30% to about 70%, about 30% to about 60%, about 30% to about 50%, about 30% to about 40%, about 35% to about 85%, about 35% to about 75%, about 35% to about 65%, about 35% to about 55%, or about 35% to about 45%, by weight of the total composition, including all values therebetween.

In one embodiment of the solid dispersion, the weight ratio of the polymer or the polymeric carrier and Compound A or a pharmaceutically acceptable salt, solvate, stereoisomer or prodrug thereof is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, or about 10:90, including all values therebetween.

In one embodiment, the solid dispersion comprises any solvent in less than about 10% by weight. In one embodiment, the solid dispersion comprises any organic solvent in less than about 8% by weight. In one embodiment, the solid dispersion comprises any organic solvent in less than: about 8%, about 7.5%, about 7%, about 6.5%, about 6%, about 5.5%, about 5%, about 4.5%, about 4%, about 3.5%, about 3%, about 2.5%, about 2%, about 1.5%, about 1%, or about 0.5% by weight, including all values therebetween.

The solid dispersion may comprise Compound A and a polymeric carrier, wherein Compound A is substantially amorphous and dispersed in a polymer matrix of the polymeric carrier. The solid dispersion comprises the polymeric carrier in an amount from about 10 parts to about 55 parts by weight based on 100 parts by weight of Compound A. In an embodiment, the solid dispersion comprises the polymeric carrier in amount from about 10 parts to about 50 parts, about 12 to about 50 parts by weight based on 100 parts by weight of Compound A. In a specific embodiment, the polymeric carrier is about 15 to about 50 parts by weight. In a more specific embodiment, the polymeric carrier is about 20 to about 50 parts by weight. In a still more specific embodiment, the polymeric carrier is about 25 to about 50 parts by weight. In a detailed embodiment, the polymeric carrier is about 25 to about 45 parts by weight.

The polymeric carrier may be chosen for any polymer that allows for Compound A to be dispersed throughout such that a solid dispersion may form. The polymeric carrier is preferably water-soluble or hydrophilic. The polymeric carrier may contain a mixture of two or more polymers. Examples of polymeric carriers include, but are not limited to, vinyl polymers and copolymers, vinyl pyrrolidine-vinyl acetate copolymer (“PVP-VA”), polyvinyl alcohols, polyvinyl alcohol-polyvinyl acetate copolymers, polyvinyl acetate phthalates (PVAP), polyvinyl pyrrolidine (“PVP”), crospovidone, poly(acrylic acid) (such as Carbomer®), acrylate and methacrylate copolymers, methylacrylic acid methyl methacrylate copolymer (such as Eudragit®), polyethylene glycols (e.g., PEG 4000, PEG 6000, PEG 8000, PEG 20000), D-α-tocopherol polyethylene glycol 1000 succinate (TPGS 1000), polyethylene-polyvinyl alcohol copolymers (PEG-PVA), polyoxyethylene-polyoxypropylene block copolymers (also referred to as poloxamers), graft copolymer comprised of polyethylene glycol, polyvinyl caprolactam and polyvinyl acetate (such as Soluplus®), cellulosic polymers, such as hydroxypropyl methyl cellulose acetate (“HPMCA”), hydroxypropyl methyl cellulose (“HPMC” or hypromellose), hydroxypropyl cellulose (“HPC”), methyl cellulose (MC), hydroxyethyl methyl cellulose, hydroxyethyl cellulose, hydroxyethyl cellulose acetate, and hydroxyethyl ethyl cellulose, hydroxypropyl methyl cellulose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), cellulose acetate trimellitate (“CAT”), hydroxypropyl methyl cellulose acetate trimellitate (“HPMCAT”), and carboxymethylcellulose acetate butyrate (“CMCAB”), chitosan, phospholipid complex and the like. The polymeric carrier may contain a mixture of two or more polymers listed above. In a further embodiment, the polymeric carrier is hydrophilic.

In a still further embodiment, the polymeric carrier is hypromellose (HPMC). In still yet a further embodiment, the hypromellose has a methoxy group substitution ratio of 19-30% and a hydroxypropyl group substitution rate of 4-12%. In a specific embodiment, the hypromellose is HPMC 2208 or HPMC 2910. In a more specific embodiment, the HPMC 2208 has a viscosity of about 100-300,000 mPa·s. In another more specific embodiment, the HPMC 2910 has a viscosity of about 3,000-10,000 mPa·s.

In a still another embodiment, the polymeric carrier is hydroxypropyl methyl cellulose acetate succinate (HPMCAS). In a specific embodiment, the HPMCAS has a viscosity of from about 2 to about 4 mPa·s.

In an embodiment, the Compound A is molecularly dispersed in the polymeric matrix. In another embodiment, the solid dispersion forms a single phase.

In another embodiment, the solid dispersion further comprises a glidant/adsorbent in addition to Compound A and the polymeric carrier at a weight content of up to 5% of the solid dispersion. In a further embodiment, the glidant/adsorbent is colloidal silica.

In a further embodiment the solid dispersion of Compound A does not comprise a surfactant.

### Methods of Manufacturing Solid Dispersions of Compound A

The solid dispersions are generally prepared by methods based on solvent evaporation. The solvent evaporation method works by dissolving the drug substance and the polymeric carrier in a suitable solvent (one co-solvent or separate solvent for each of the two components) to form a feed solution, and then the feed solution may be subject to procedures to remove the solvent (e.g., spray-dried) to form the solid dispersion. A generalized process of preparing a solid dispersion comprises:
(a) dissolving Compound A and at least one polymeric carrier in a suitable co-solvent or two or more separate solvents and
(b) evaporating the co-solvent or separate solvents to form the solid dispersion.

The evaporation of the solvent in step (b) is performed by spray-drying, melt extrusion, freeze drying, rotary evaporation, drum drying, fluid bed granulation or other solvent removal processes. Art-known methods can be used to prepare the Compound A solid dispersion and these methods would be familiar to a person having ordinary skill in the art for making solid dispersions. In an embodiment, the solid dispersion are prepared by conventional spray-drying techniques. In another embodiment, the solvent evaporation method used for preparing is reduced pressure evaporation. In still another embodiment, the solid evaporation used is fluid bed granulation. In other embodiments, other techniques known in the art may be used for preparing the solid dispersion such as hot-melt extrusion, kneading, co-milling, simple fusion, microwave induced fusion, KinetiSol® dispersing, simple solvent evaporation, lyophilization, electrostatic spinning, fluid-bed coating, solvent-melt, supercritical carbon dioxide and co-precipitation processes.

Example processes of solvent evaporation methods as well as non-solvent evaporation methods for manufacturing Compound A, solid dispersions by spray-drying and by hot-melt extrusion, are briefly described below.

### 1. Solid Dispersions by Spray-Drying

A suitable co-solvent or co-solvent system for both Compound A and the polymeric carrier, when available can be used. In the case of co-solvent, a pre-weighed amount of Compound A is slowly added into the spray solution while mixing, followed by slow addition of the polymeric carrier (for example, HPMC) to yield a feed solution. Alternatively, separate solvents can be used for Compound A and the polymeric carrier and combined to give the feed solution

The resulting feed solution is spray dried using a standard pharmaceutical grade spray dryer, such as MS-150. Following completion of spray drying, the Spray-Dried Dispersion (SDD) may be dried in an oven until the residual acetone is below ICH guidelines, 5000 ppm. The dried SDD is then transferred into appropriate containers with desiccants to protect from moisture. This SDD can be further formulated by e.g., dry granulation and blending as described below.

Suitable solvents for spray-drying are a solvent or mixture of solvents in which both Compound A and the polymeric carrier have adequate solubility (solubility greater than 1 mg/mL). Separate solvents may be used if each component of the solid dispersion require different solvents to obtain the desired solubility. The solvent may be volatile with a boiling point of 150 °C or less. In addition, the solvent should have relatively low toxicity and be removed from the dispersion to a level that is acceptable to The International Committee on Harmonization (“ICH”) guidelines. Removal of solvent to this level may require a subsequent processing step, such as tray drying. Examples of suitable solvents include, but are not limited to, alcohols, such as methanol, ethanol, n-propanol, isopropanol (“IPA”) and butanol; ketones, such as acetone, methyl ethyl ketone (“MEK”) and methyl isobutyl ketone; esters, such as ethyl acetate (“EA”) and propyl acetate; and various other solvents, such as tetrahydrofuran (“THF”), acetonitrile (“ACN”), methylene chloride, toluene and 1,1,1-trichloroethane; and any combinations thereof. Lower volatility solvents, such as dimethyl acetate or dimethylsulfoxide (“DMSO”), may be used. Mixtures of solvents with water may also be used, so long as the polymeric carrier is sufficiently soluble to make the spray drying process practicable. Generally, due to the hydrophobic nature of low solubility drugs, non-aqueous solvents may be used, meaning the solvent comprises less than about 10 weight % water.

In certain embodiments, the polymeric carrier is HPMC and the suitable solvent for spray-drying is separate solvent system made of ethanol and water, with ethanol used for Compound A and water for the HPMC. In a more specific embodiment, water is used in a 100-140-fold excess of HPMC by weight, and ethanol is used a 5-12-fold excess of Compound A.

Suitable spray drying parameters are known in the art, and it is within the knowledge of a skilled artisan in the field to select appropriate parameters for spray drying. For instance, the target feed concentration is generally about 1 to about 50% and a viscosity of about 1 to about 300 cP, or about 1 to about 80 cP, or about 4 to 60 cP. The inlet temperature of the spray dry apparatus is typically about 50-190 °C, while the outlet temperature is about 30-90 °C. A two fluid nozzle and/or hydraulic pressure nozzle can be used to spray-dry Compound A. The spray-dried material typically has a particle size (D₉₀) of less than about 500 m, about 200 m, about 150 m, about 100 m, about 50 m, or about 25 m, in some instances. A milling step may be used if desired to further reduce the particle size, in additional instances.

### 2. Solid Dispersions by Hot-Melt Extrusion

Solid Dispersions of amorphous Compound A can be formulated using a hot-melt extrusion process (referred to herein as formulation, HMIE solid dispersion formulation, or HME formulation) comprising hot-melt extrusion, milling, blending, and optionally tableting. Multiple hot-melt extrusion and milling batches may be combined for blending and tableting to make larger batch sizes.

The amounts of materials used for making the solid dispersions are within the weight percentages or ratios ranges in any of the embodiments described in this disclosure.

### [Hot-Melt Extrusion and Milling]

Suitable amounts of Compound A and one or more solubilizing agents, such as copovidone as a non-limiting example, are added to a blender and blended for about 5 minutes. Non-limiting examples of blending/mixing equipment that can be used include a diffusion mixer (for example, V-blender or bin-blender) or a convection mixer (for example, a vertical high intensity mixer). In another embodiment, a V-blender is used for the blending/mixing. The contents are then screen sieved and then transferred back to the blender and blended for about 10 minutes.

A suitable amount of one or more solubilizing agents (such as polyethylene glycol 400 (PEG400)) are weighed in a suitable container. An additional about 20 g of PEG 400 is used for setting up the extruder, and this additional about 20 g of PEG 400 was not part of the solid dispersion. An extruder is set up using appropriate set points and using about 20 g of PEG 400 to adjust the flow rate.

The blend is added to a feed hopper and the extrusion process is initiated. The extrusion parameters are adjusted and monitored as necessary. The resulting pelletized extrusion are collected and can be weighed and placed in a suitable container.

### Pharmaceutical Compositions, Solid Dosage Forms

In another aspect, the present invention provides a pharmaceutical composition comprising Compound A solid dispersion described above and pharmaceutically acceptable excipients.

The pharmaceutical composition comprises
a) from about 20% to about 50% w/w of Compound A solid dispersion,
b) from about 25% to about 70% w/w of filler/binder,
c) from about 4.0% to about 20% w/w of disintegrant,
d) from about 0.25% to about 5.0% w/w of glidant/adsorbent and
e) from about 0.5% to about 2.5% w/w of lubricant;
where the Compound A solid dispersion is as described above. The Compound A solid dispersion is an amorphous solid dispersion. More specifically, the Compound A solid dispersion has Compound A dispersed substantially amorphously in a polymer matrix of a hydrophilic polymeric carrier in an amount of from about 64.5% to about 91% w/w Compound A, the weight percentage being based on the combined weight of the Compound A and the hydrophilic polymeric carrier.

The filler/binder in the pharmaceutical composition can be selected from the group consisting of starch, saccharides, disaccharides, sucrose, lactose, lactose monohydrate, polysaccharides, cellulose, cellulose ethers, hydroxypropylcellulose, sugar alcohols, xylitol, sorbitol, maltitol, microcrystalline cellulose, calcium or sodium carbonate, dicalcium phosphate, compressible sugars, dibasic calcium phosphate dehydrate, mannitol, tribasic calcium phosphate, and any combination thereof.

The disintegrant in the pharmaceutical composition can be selected from the group consisting of starch, sodium starch glycolate, croscarmellose sodium, crospovidone, microcrystalline cellulose, modified corn starch, sodium carboxymethyl starch, povidone, pregelatinized starch, alginic acid and any combination thereof.

In an embodiment, the pharmaceutical composition comprises c) from about 4.5% to about 15% w/w of disintegrant. In a further embodiment, the pharmaceutical composition comprises c) from about 4.9% to about 12% w/w of disintegrant. In a still further embodiment, the pharmaceutical composition comprises c) from about 4.9% to about 10% w/w of disintegrant.

The glidant/adsorbent in the pharmaceutical composition can be selected from the group consisting of colloidal silicon dioxide, talc, fumed silica, starch, starch derivatives, bentonite and any combination thereof.

The lubricant in the pharmaceutical composition can be selected from the group consisting of magnesium stearate, stearic acid, silica, fats, calcium stearate, polyethylene glycol, sodium stearyl fumarate, or talc, lauric acid, oleic acid, C8/C10 fatty acid and any combination thereof.

Examples of the polymeric carrier include, but are not limited to poly vinyl pyrrolidine-vinyl acetate copolymer (“PVP-VA”), polyvinyl alcohols, polyvinyl alcohol-polyvinyl acetate copolymers, polyvinyl acetate phthalates (PVAP), polyvinyl pyrrolidine (“PVP”), polyethylene glycols (e.g., PEG 4000, PEG 6000, PEG 8000, PEG 20000), D-α-tocopherol polyethylene glycol 1000 succinate (TPGS 1000), polyethylene-glycol polyvinyl alcohol copolymers (PEG-PVA), cellulosic polymers, such as hydroxypropyl methyl cellulose acetate (“HPMCA”), hydroxypropyl methyl cellulose (“HPMC” or hypromellose), hydroxypropyl cellulose (“HPC”), methyl cellulose (MC), hydroxyethyl methyl cellulose, hydroxyethyl cellulose, hydroxyethyl cellulose acetate, and hydroxyethyl ethyl cellulose, hydroxypropyl methyl cellulose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), cellulose acetate trimellitate (“CAT”), hydroxypropyl methyl cellulose acetate trimellitate. Mixtures of two or more polymers listed above can be used as the hydrophilic polymeric carrier. In a specific embodiment, the hydrophilic polymeric carrier is hypromellose (HPMC). In a further specific embodiment, the hypromellose has a methoxy group substitution ratio of 19-30% and a hydroxypropyl group substitution rate of 4-12%. In a specific embodiment, the hypromellose is HPMC 2208 or HPMC 2910. In a more specific embodiment, the HPMC 2208 has a viscosity of about 2,500-10,000 mPa·s. In another more specific embodiment, the HPMC 2910 has a viscosity of about 100-300,000 mPa·s. In a further yet specific esmbodiment, the polymeric carrier is hydroxypropyl methyl cellulose acetate succinate (HPMCAS).

In another embodiment, the Compound A solid dispersion is formed by spray-drying. In a further specific embodiment, the spray-dried Compound A solid dispersion comprises hypromellose as the hydrophilic polymeric carrier.

In yet another embodiment, the pharmaceutical composition is a solid oral dosage form in the form of a tablet, a pill, a caplet, granules, a modified release formulation, a controlled release formulation, a sustained release formulation, an immediate release formulation, or a powder.

In still another embodiment is a pharmaceutical composition or the spray-dried formulation described herein, wherein the pharmaceutical composition or formulation provides improved pharmacokinetics (PK) properties as compared to a reference tablet formulation of Compound A that is not in the form of amorphous solid dispersion, but crystalline. In a specific embodiment, the pharmaceutical composition is a tablet or a capsule, either of which provides, in human or a test animal, 2-3 fold increase in drug exposure (i.e., Cₘₐₓ) as compared to a reference tablet formulation comprising identical amount of Compound A but not comprising an amorphous solid dispersion of Compound A. For Compound A pharmaceutical compositions or formulations comprising an non-free base form of Compound A, what is meant by the identical amount is the equivalent amount in the free base anhydrate form for comparison. In a further embodiment, the drug exposure (Cₘₐₓ) comparison is based on beagle dogs, i.e. the test animal is a beagle dog.

In another specific embodiment, the pharmaceutical composition or the spray-dried formulation described herein, wherein the pharmaceutical composition is a tablet or a capsule, either of which provides, in human or a test animal, 2-3-fold increase in area under the curve (AUC) as compared to a reference tablet formulation comprising identical amount of Compound A but not comprising an amorphous solid dispersion of Compound A (i.e., the reference tablet has crystalline Compound A). For Compound A pharmaceutical compositions or formulations comprising an non-free base form of Compound A, what is meant by the identical amount is the equivalent amount in the free base anhydrate form for comparison. In a further embodiment, the area under the curve comparison is based on beagle dogs, i.e. the test animal is a beagle dog.

In yet another embodiment, the pharmaceutical composition or the spray-dried formulation described herein, provides an immediate release of Compound A, wherein the immediate-release pharmaceutical composition is a tablet or a capsule. In a still further embodiment, the immediate-releasing pharmaceutical composition comprises c) from about 4.5% to about 15% w/w of disintegrant. In still further an embodiment, the pharmaceutical composition comprises c) from about 4.9% to about 12% w/w of disintegrant. In an even further yet embodiment, the pharmaceutical composition comprises c) from about 4.9% to about 10% w/w of disintegrant.

In yet another aspect, the present invention provides a solid oral formulation comprising Compound A solid dispersion providing improved pharmacokinetics (PK) properties as compared to a reference tablet formulation of Compound A comprising the free base form of Compound A in an identical amount, but not in the form of amorphous solid dispersion. For Compound A solid oral formulations comprising an non-free base form of Compound A, what is meant by the identical amount is the equivalent amount in the free base anhydrate form for comparison. The Compound A solid dispersion in the solid oral formulation comprises substantially amorphous Compound A dispersed in a polymer matrix of hydroxypropyl methyl cellulose in an amount of from about 64.5% to about 91% w/w Compound A based on the combined weight of the Compound A and hydroxypropyl methyl cellulose. In a particular embodiment, the solid oral formulation is a tablet.

In an embodiment, the solid oral formulation is a pharmaceutical composition providing, in human or a test animal, 2-3 fold increase in drug exposure (i.e., Cₘₐₓ) as compared to a reference tablet formulation comprising identical amount of Compound A but not comprising an amorphous solid dispersion of Compound A. In a specific embodiment, the drug exposure (Cₘₐₓ) comparison is based on beagle dogs, i.e. the test animal is a beagle dog. In a further specific embodiment, the solid oral formulation is a tablet or a capsule.

In another embodiment, the solid oral formulation is a pharmaceutical composition providing, in human or a test animal, 2-3-fold increase in area under the curve (AUC) as compared to a reference tablet formulation comprising identical amount of Compound A but not comprising an amorphous solid dispersion of Compound A. In a specific embodiment, the area under the curve comparison is based on beagle dogs, i.e. the test animal is a beagle dog. In a further specific embodiment, the solid oral formulation is a tablet or a capsule. In a still further embodiment, the solid oral formulation is a tablet comprises c) from about 4.9% to about 10% w/w of disintegrant.

In yet another embodiment, the Compound A solid dispersion of the solid oral formulation is formed by solvent evaporation methods such as spray-drying and fluid bed granulation.

In some embodiments, compositions described herein (including, for example, the pharmaceutical compositions described herein) are formulated as a tablet comprising a solid dispersion. Such a tablet may be referred to herein as a “solid dispersion tablet”. In certain embodiments, compositions described herein (including, for example, the pharmaceutical compositions described herein) are formulated as a tablet comprising a spray-dried solid dispersion. Such a tablet may be referred to herein as a “spray-dried solid dispersion tablet”.

In some embodiments, solid dosage forms, e.g., tablets, effervescent tablets, and capsules, are prepared by mixing the Compound A with one or more pharmaceutical excipients to form a bulk blend composition. When referring to these bulk blend compositions as homogeneous, it is meant that the particles of the Compound A are dispersed evenly throughout the composition so that the composition may be readily subdivided into equally effective unit dosage forms, such as tablets, pills, and capsules. The individual unit dosages may also include film coatings, which disintegrate upon oral ingestion or upon contact with diluent. These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, or (6) fusion.

Dry Granulation and Blending of Spray-Dried Solid Dispersion with Intragranular and Extragranular Excipients

The technology of employing dry granulation and blending to the solid dispersion of Compound A and the pharmaceutical composition comprising the solid dispersion to manufacture tablets would be familiar to a person skilled in the art. The procedure is briefly outlined below.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of Compound A from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more further excipients.

The spray-dried solid dispersion and intra-granular excipients can be dispensed into appropriate containers. The intra-granular excipients employed are one or more lubricants (such as sodium stearyl fumarate as a non-limiting example); one or more glidant/adsorbents (such as colloidal silicon dioxide as a non-limiting example); one or more disintegrants (such as croscarmellose sodium as a non-limiting example); and one or more fillers/binders (such as mannitol and microcrystalline cellulose as non-limiting examples). The spray-dried solid dispersion and intra-granular excipients are added to a blender of an appropriate size and blended. The blend is passed through a sieve (such as a comil) to improve blend uniformity and remove large particles. This blend is further blended and then discharged into appropriate container.

The blend is then dry granulated to result in ribbons using an appropriate roller compactor, such as TFC-220 roller compactor or others, and by using selected process parameters (roll type, RPM and roll compaction force). The resulting ribbons are milled (which can be done by using a comil, for example) to result in a free flowing granulation.

Appropriate quantities of the extra-granular excipients are then added. The extra granular excipients include one or more lubricants (such as sodium stearyl fumarate as a non-limiting example); one or more glidant/adsorbents (such as colloidal silicon dioxide, and croscarmellose sodium as a non-limiting example); to the granulation and blend to obtain the blend for tablet compression.

The granulated spray-dried solid dispersion can then be tableted and packaged as described below.

### Tableting

The granulated spray-dried solid dispersion can be tableted with a rotary tablet press. The rotary tablet press is set up to yield target tablet weight, hardness, and friability. Weight and hardness can be monitored at initial startup and at about 15 minute intervals. Metal check and weight sorting can be performed for the tablets.

### Pharmaceutical Compositions for Capsule

In yet another aspect, the present invention provides a pharmaceutical composition for capsule comprising Compound A solid dispersion described above and pharmaceutically acceptable excipients.

The pharmaceutical composition comprises:
a) from about 50% to about 97.5% w/w of Compound A solid dispersion;
b) from about 0% to about 48% w/w of filler/binder;
c) from about 0% to about 20% w/w of disintegrant;
d) from about 0% to about 4.0% w/w of glidant/adsorbent; and
e) from about 0.2% to about 4.0% w/w of lubricant.

### Manufacturing Capsules

Applying pharmaceutical technology to the solid dispersion of Compound A and the pharmaceutical composition comprising the solid dispersion to form capsules would be a process familiar to a person skilled in the art. The procedure is briefly outlined below.

In some embodiments, a capsule is prepared by placing the bulk blend of the solid formulation inside of a capsule. In some embodiments, the solid formulation is placed in a soft gelatin capsule. In some embodiments, the formulation is placed in a hard gelatin capsule. In some embodiments, the solid formulation is placed in standard gelatin capsules or non-gelatin capsules such as capsules comprising HPMC. In some embodiments, the solid formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

The capsule can be any shape or size. The shape and size of the capsule can vary in accordance with the method of preparation and the planned use of the capsule. Hard shell capsule sizes are designated by convention as (000), (00), (0), (1), (2), (3), (4), and (5), with a larger number corresponding to a smaller size. For example, one teaspoon can fill approximately seven size “0” capsules and about five size “00” capsules. Size “00” capsules are generally the largest size utilized for human consumption. Soft gel capsule sizes range from 1 to 120, with the particular size depending on the desired shape and other characteristics of the product. Typically, shapes include, but are not limited to, round, oval, oblong, tube, and special shapes, for example, animals, stars, hearts, or squares. For example, capsules can be oval-shaped and be size 2, 3, 4, 5, 6, 7, 7.5, 8.5, 10, 12, 16, 20, 30, 40, 60, 65, 80, or more. Capsules can be, for example, oblong-shaped and be size 3, 4, 5, 6, 8, 9.5, 11, 12, 14, 16, 20, 22, 24, or more. Other exemplary capsules include round capsules, for example, size 1, 2, 3, 4, 5, 6, 7, 9, 15, 20, 28, 40, 90, or more, and tube capsules, for example, size 5, 6, 8, 17.5, 30, 45, 55, or 120. Capsules can be any color and have any amount of transparency, for example, the capsules can be, but are not limited to, opaque, translucent, or pearlescent capsules. In some embodiments, the solid formulation is filled into a hard gelatin capsule. In some embodiments, the capsule is a two-piece hard gelatin capsule. In some embodiments, the capsule is sealed. In some embodiments, the capsule is unsealed.

### Therapeutic Use and Dosage

In still another aspect, provided herein are methods for treating a patient by administering the pharmaceutical composition described above. In some embodiments, provided herein is a method of inhibiting the activity of tyrosine kinase(s), such as FLT3, SYK, JAK and mutant c-KIT(D815H, D816V), or of treating a disease, disorder, or condition, which would benefit from inhibition of tyrosine kinase(s), such as FLT3, SYK, JAK and mutant c-KIT(D815H, D816V), in a mammal, which includes administering to the mammal a therapeutically effective amount of Compound A, or its pharmaceutically acceptable salt, its hydrate or a hydrate of its pharmaceutically acceptable salt.

In some embodiments, the pharmaceutical composition is administered to a human.

In some embodiments, the pharmaceutical composition is orally administered.

In other embodiments, the pharmaceutical composition is used for the formulation of a medicament for the inhibition of tyrosine kinase activity.

In still yet another aspect, provided herein is a method of treating cancer in a mammal comprising administering to the mammal a pharmaceutical composition or formulation described herein comprising amorphous solid dispersion of Compound A. In some embodiments, the cancer is a B cell malignancy. In some embodiments, the cancer is leukemia. In still further embodiments, the leukemia is acute myeloid leukemia.

In one embodiment, the pharmaceutical composition is a solid dosage form containing a therapeutically effective amount of Compound A. In some embodiments, the solid dosage form can be administered by a variety of routes, depending upon the treatment desired and upon the area to be treated. In a further embodiment, the treatment is for cancer. In a still further embodiment, the cancer is leukemia. In a yet another further embodiment, the leukemia is acute myeloid leukemia. In some embodiments, administration is oral. Oral administration can include a dosage form formulated for once-daily or twice-daily (BID) administration.

The pharmaceutical compositions comprising Compound A solid dispersion can be formulated in a unit dosage form, each dosage containing from about 1 to about 1,000 mg (1 g), more usually about 5 mg to about 100 mg of Compound A, the active ingredient. The term “unit dosage form” refers to physically discrete units suitable as unitary dosages for human subjects and other patients, each unit containing a predetermined quantity of active ingredient (i.e., Compound A or a pharmaceutically acceptable salt, amorphous form, a hydrate thereof etc.) calculated to produce the desired therapeutic effect, in association with a suitable pharmaceutical excipient.

In some embodiments, the pharmaceutical composition is formulated as a 1 mg, 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg dosage form. In some embodiments, the solid formulation is formulated as a 10 mg, 20 mg, or 80 mg dosage form.

In some embodiments, the compositions provided herein contain from about 1 mg to about 50 mg of the active ingredient, Compound A. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient. In some embodiments, the compositions provided herein contain about 10 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The daily dosage of Compound A or its hydrate, a pharmaceutically acceptable salt of Compound A, a hydrate of the salt thereof can be varied over a wide range from 1.0 to 10,000 mg per adult human per day, or higher, or any range therein. In some embodiments, for oral administration, the pharmaceutical compositions can be provided in the form of tablets containing about 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.1 mg/kg to about 1000 mg/kg of body weight per day, or any range therein. In some embodiments, the range is from about 0.5 to about 500 mg/kg of body weight per day, or any range therein. In some embodiments, from about 1.0 to about 250 mg/kg of body weight per day, or any range therein. In some embodiments, from about 0.1 to about 100 mg/kg of body weight per day, or any range therein. In some embodiments, the range is from about 0.1 to about 50.0 mg/kg of body weight per day, or any amount or range therein. In some embodiments, the range is from about 0.1 to about 15.0 mg/kg of body weight per day, or any range therein. In some embodiments, the range is from about 0.5 to about 7.5 mg/kg of body weight per day, or any amount or range therein. Pharmaceutical compositions containing Compound A, a hydrate thereof, a pharmaceutically acceptable salt thereof, or a hydrate of a pharmaceutically acceptable salt thereof can be administered on a regimen of 1 to 4 times per day or in a single daily dose.

The present disclosure also relates to methods for treating cancer, comprising administering any one of the pharmaceutical compositions as disclosed herein to a subject in need thereof. The present disclosure also relates to methods for treating cancer, comprising administering any one of the amorphous forms of Compound A as disclosed herein to a subject in need thereof.

In a specific embodiment, the cancer is a leukemia such as acute myelogenous leukemia (AML), chronic myelogenous leukemia (CML), acute lymphocytic leukemia (ALL), chronic lymphocytic leukemia, CLL), acute promyelocytic leukemia (APL), hairy cell leukemia, or chronic neutrophilic leukemia (CNL). In a specific embodiment, the leukemia is AML. In another specific embodiment, the leukemia comprises leukemia cancers having a mutation in the FLT3 gene. In another specific embodiment, the FLT3 gene has an internal tandem duplication (ITD) mutation. In another specific embodiment, the FLT3 gene has at least one FLT3 mutation selected from F691L, D835Y, D835F, D835I, D835H, D835V and D835A.

### Enumerated Embodiments

1. A solid dispersion comprising: Compound A; and
a polymeric carrier in an amount of from about 10 parts to about 55 parts by weight based on 100 parts by weight of Compound A;
wherein the Compound A is substantially amorphous and dispersed in a polymer matrix of the polymeric carrier within the solid dispersion.
2. The solid dispersion according to embodiment 1, wherein the polymeric carrier is in an amount from about 10 parts to about 50 parts, specifically about 12 parts to 50 parts, more specifically about 15 parts to 50 parts, and still more specifically about 20 parts to about 50 parts by weight based on 100 parts by weight of Compound A.
3. The solid dispersion according to embodiment 1, wherein the polymeric carrier is selected from the group consisting of Poly vinyl pyrrolidine-vinyl acetate copolymer (PVP-VA), polyvinyl alcohols, polyvinyl alcohol-polyvinyl acetate copolymers, polyvinyl acetate phthalates (PVAP), polyvinyl pyrrolidine (PVP), polyethylene glycols (e.g., PEG 4000, PEG 6000, PEG 8000, PEG 20000), D-α-tocopherol polyethylene glycol 1000 succinate (TPGS 1000), polyethylene glycol-polyvinyl alcohol copolymers (PEG-PVA), cellulosic polymers, such as hydroxypropyl methyl cellulose acetate (“HPMCA”), hydroxypropyl methyl cellulose (“HPMC” or hypromellose), hydroxypropyl cellulose (“HPC”), methyl cellulose (MC), hydroxyethyl methyl cellulose, hydroxyethyl cellulose, hydroxyethyl cellulose acetate, and hydroxyethyl ethyl cellulose, hydroxypropyl methyl cellulose acetate succinate (“HPMCAS”), hydroxypropyl methyl cellulose phthalate (“HPMCP”), carboxymethylethyl cellulose (“CMEC”), cellulose acetate phthalate (“CAP”), cellulose acetate succinate (“CAS”), hydroxypropyl methyl cellulose acetate phthalate (“HPMCAP”), D-α-tocopherol polyethylene glycol succinate (TPGS), chitosan, phospholipid complex and any mixture thereof.
4. The solid dispersion according to embodiment 3, wherein the polymeric carrier is a hydrophilic carrier.
5. The solid dispersion according to embodiment 2, wherein the wherein the polymeric carrier is one or more cellulose derivative selected from the group consisting of hydroxypropyl methyl cellulose (HPMC), hydroxypropyl methyl cellulose phthalate (HPMCP), hydroxypropyl methyl cellulose acetate succinate (HPMCAS), methyl cellulose, ethyl cellulose, hydroxyethyl cellulose, hydroxypropyl cellulose, carboxymethyl cellulose, cellulose acetate and cellulose acetate phthalate.
6. The solid dispersion according to embodiment 1, wherein the solid dispersion is formed by a solvent evaporation method.
7. The solid dispersion according to embodiment 1, wherein the solvent evaporation method is spray drying or fluid bed granulation.
8. The solid dispersion according to embodiment 1, wherein the solid dispersion further comprises a glidant/adsorbent.
9. A pharmaceutical composition comprising
   a) from about 20% to about 50% w/w of Compound A solid dispersion;
   b) from about 25% to about 70% w/w of filler/binder;
   c) from about 4.0% to about 20% w/w of disintegrant;
   d) from about 0.25% to about 5.0% w/w of glidant/adsorbent; and
   e) from about 0.5% to about 2.5% w/w of lubricant;
wherein Compound A is a compound of Compound A or a pharmaceutically acceptable salt thereof:

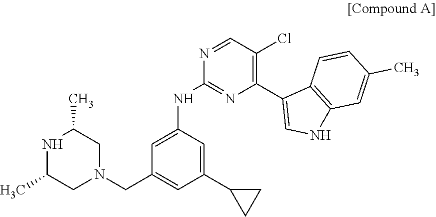

wherein the Compound A solid dispersion comprises substantially amorphous Compound A dispersed in a polymer matrix of a hydrophilic polymeric carrier in an amount of from about 64.5% to about 91% w/w Compound A based on the combined weight of the Compound A and the hydrophilic polymeric carrier.
10. The pharmaceutical composition according to embodiment 9, wherein the pharmaceutical composition is a solid oral dosage form selected from the group consisting of tablets, powders, granules, caplets and pills.
11. The pharmaceutical composition according to embodiment 10, wherein the solid oral dosage form is a tablet.
12. The pharmaceutical composition according to embodiment 9, wherein the hydrophilic polymeric carrier is hydroxypropyl methyl cellulose.
13. The pharmaceutical composition according to embodiment 9, wherein the filler/binder is selected from the group consisting of starch, saccharides, disaccharides, sucrose, lactose, lactose monohydrate, polysaccharides, cellulose, cellulose ethers, hydroxypropylcellulose, sugar alcohols, xylitol, sorbitol, maltitol, microcrystalline cellulose, calcium or sodium carbonate, dicalcium phosphate, compressible sugars, dibasic calcium phosphate dehydrate, mannitol, tribasic calcium phosphate, and any combination thereof.
14. The pharmaceutical composition according to embodiment 9, wherein the disintegrant is selected from the group consisting of starch, sodium starch glycolate, croscarmellose sodium, crospovidone, microcrystalline cellulose, modified corn starch, sodium carboxymethyl starch, povidone, pregelatinized starch, alginic acid and any combination thereof.
15. The pharmaceutical composition according to embodiment 9, wherein the glidant/adsorbent is selected from the group consisting of colloidal silicon dioxide, talc, fumed silica, starch, starch derivatives, bentonite and any combination thereof.
16. The pharmaceutical composition according to embodiment 9, wherein the lubricant is selected from the group consisting of magnesium stearate, stearic acid, silica, fats, calcium stearate, polyethylene glycol, sodium stearyl fumarate, or talc, lauric acid, oleic acid, C8/C10 fatty acid and any combination thereof.
17. The pharmaceutical composition according to embodiment 9, wherein the Compound A solid dispersion is formed by spray drying.
18. The pharmaceutical composition according to embodiment 9, comprises c) from about 4.5% to about 15% w/w of disintegrant, more specifically, c) from about 4.9% to about 12% w/w of disintegrant, still more specifically, c) from about 4.9% to about 10% w/w of disintegrant.
19. The pharmaceutical composition according to embodiment 11, wherein the tablet provides about 2-fold to about 3-fold increase in drug exposure over a reference tablet comprising an identical amount of Compound A but not comprising an amorphous solid dispersion of Compound A.
20. The pharmaceutical composition according to embodiment 20, wherein the drug exposure comparison is based on beagle dogs.
21. The pharmaceutical composition according to embodiment 11, wherein the tablet provides about 2-fold to about 3-fold increase in area under the curve (AUC) over a reference tablet comprising an identical amount of Compound A but not comprising an amorphous solid dispersion of Compound A.
22. The pharmaceutical composition according to embodiment 21, wherein the area under the curve comparison is based on beagle dogs.
23. A pharmaceutical composition for capsule comprising
   a) from about 50% to about 97.5% w/w of Compound A solid dispersion;
   b) from about 0% to about 48% w/w of filler/binder;
   c) from about 0% to about 20% w/w of disintegrant;
   d) from about 0% to about 4.0% w/w of glidant/adsorbent; and
   e) from about 0.2% to about 4.0% w/w of lubricant;
wherein Compound A is a Compound A or a pharmaceutically acceptable salt thereof:

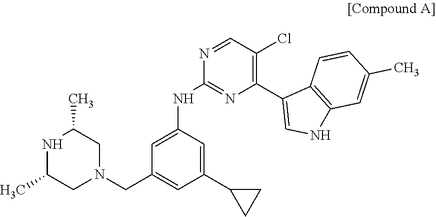

and
wherein the Compound A solid dispersion comprises substantially amorphous Compound A dispersed in a polymer matrix of a hydrophilic polymeric carrier in an amount of from about 64.5% to about 91% w/w Compound A based on the combined weight of the Compound A and the hydrophilic polymeric carrier.
24. The pharmaceutical composition according to embodiment 9, wherein the pharmaceutical composition comprises from about 4.9% to about 12% w/w of disintegrant.
25. A solid oral formulation comprising from about 20% to about 50% w/w of Compound A solid dispersion,
wherein the oral formulation provides about 2-fold to about 3-fold increase in drug exposure over a reference tablet comprising an identical amount of Compound A but not comprising an amorphous solid dispersion of Compound A,
wherein the Compound A solid dispersion comprises substantially amorphous Compound A dispersed in a polymer matrix of hydroxypropyl methyl cellulose in an amount of from about 64.5% to about 91% w/w Compound A based on the combined weight of the Compound A and hydroxypropyl methyl cellulose.
26. A solid oral formulation comprising from about 20% to about 50% w/w of Compound A solid dispersion,
wherein the oral formulation provides about 2-fold to about 3-fold increase in area under the curve over a reference tablet comprising an identical amount of Compound A but not comprising an amorphous solid dispersion of Compound A, and
wherein the Compound A solid dispersion comprises substantially amorphous Compound A dispersed in a polymer matrix of hydroxypropyl methyl cellulose in an amount of from about 64.5% to about 91% w/w Compound A based on the combined weight of the Compound A and hydroxypropyl methyl cellulose.
27. The solid oral form according to embodiment 25 or claim 26, wherein the Compound A solid dispersion is formed by spray drying.
28. A solid oral formulation comprising from about 20% to about 50% w/w of Compound A solid dispersion, and
wherein the Compound A solid dispersion comprises substantially amorphous Compound A dispersed in a polymer matrix of hydroxypropyl methyl cellulose in an amount of from about 64.5% to about 91% w/w Compound A based on the combined weight of the Compound A and hydroxypropyl methyl cellulose.
29. The solid oral formulation according to embodiment 26 or 28, wherein the solid oral formulation further comprises from about 4.9% to about 12% w/w of disintegrant.
30. The solid oral formulation according to embodiment 29, wherein the solid oral formulation further comprises microcrystalline cellulose and mannitol.
31. A method of treating cancer in a mammal comprising administering to the mammal a therapeutically effective amount of a pharmaceutical composition or formulation described herein comprising amorphous solid dispersion of Compound A.
32. The method according to embodiment 31, wherein the cancer is leukemia.
33. The method according to embodiment 32, wherein the leukemia is acute myeloid leukemia.
34. The method according to embodiment 31, wherein the pharmaceutical composition is a capsule.
35. The method according to embodiment 31, wherein the pharmaceutical composition or formulation is administered in combination with another therapeutic agent.

### EXAMPLES

For illustrative purposes, the following Examples are included. However, it is to be understood that these Examples do not limit the invention and are only meant to suggest a method of practicing the invention. Persons skilled in the art will recognize that the chemical reactions described may be readily adapted to prepare the compounds described herein, and alternative methods for preparing the compounds are deemed to be within the scope of this invention. For example, the synthesis of the compounds described herein may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing the compounds described herein. Persons skilled in the art will also recognize that the solid dispersions and compositions described may be readily adapted to prepare other dispersions and compositions, and alternative methods for preparing the dispersions and compositions, as well as alternative compositions are deemed to be within the scope of this invention.

### Experimental Example 1: Analysis of Amorphous Solid Dispersions of Compound A

Amorphous solid dispersions of Compound A were prepared using spray-drying in this example. Crystalline Compound A monohydrate of the free base form was dissolved in ethanol as indicated in Table 1 below and the polymeric carrier, HPMC 2910 (Anycoat C, Lotte Fine Chemical, Korea; 4.5 mPa·s, methoxy content 28-30%, hydroxypropoxy content 7-12%) was dissolved in water as indicated in Table 1 below. The resultant aqueous solution and the ethanol solution were then combined 3200:800 to prepare the spray-dry feed solution. The feed solution was sprayed with Mini Spray Dryer B-290 (Buchi, Switzerland). Briefly, the spray-dryer was pre-heated with the inlet and outlet temperatures set at 80 °C and 37 °C, respectively. At the end of the pre-heating, spraying of the feed started with the product temperature set at 35 °C at a pump setting of 8-10 rpm accompanied by magnetic stirring at 150 rpm to prevent precipitation. The spray-dried material was then collected and dried in a chamber at 60 °C for 2 hours.

For comparison, crystalline Compound A monohydrate was used unprocessed as a non-dispersed, crystalline solid formulation of Compound A in Comparative Example 1, while a polymeric carrier-free sprayed formulation of Compound A was prepared in Comparative Example 2, and a spray-dried dispersion with excess polymeric carrier was prepared in Comparative Example 3. The resulting spray-dried example compositions as well as the compositions of comparative example formulations are given below in Table 1.

TABLE 1
Compositions of the formulations

| | Compound A | | | | Final |
|---|---|---|---|---|---|
| | Monohydrate | HPMC | Water | Ethanol | Weight |
| | (mg) | (mg) | (mg) | (mg) | (mg) |
| Example 1 | 100.0 | 25.0 | 3200 | 800 | 125.0 |
| Example 2 | 100.0 | 10.0 | 3200 | 800 | 110.0 |
| Example 3 | 100.0 | 50.0 | 3200 | 800 | 150.0 |
| Comp. Ex. | 100.0 | 0.0 | 0.0 | 0.0 | 100.0 |
| 1 | | | | | |
| Comp. Ex. | 100.0 | 0.0 | 3200 | 800 | 100.0 |
| 2 | | | | | |
| Comp. Ex. | 100.0 | 60.0 | 3200 | 800 | 160.0 |
| 3 | | | | | |

The example and comparative example formulations were evaluated for physical appearance (scanning electron microscopy), solubility, crystallinity (X-ray diffractometry and differential scanning calorimetry) to determine their amorphous character and improvement in solubility.

### 1. Physical Appearance

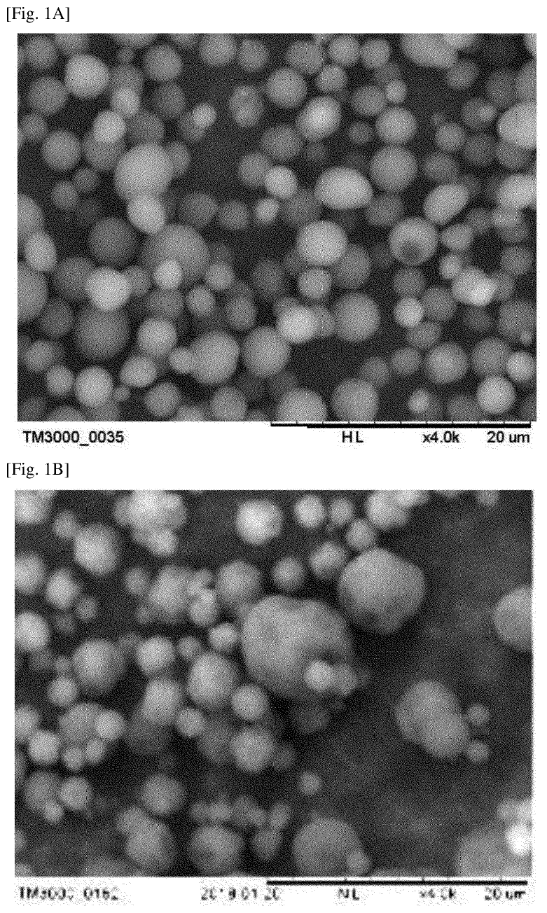

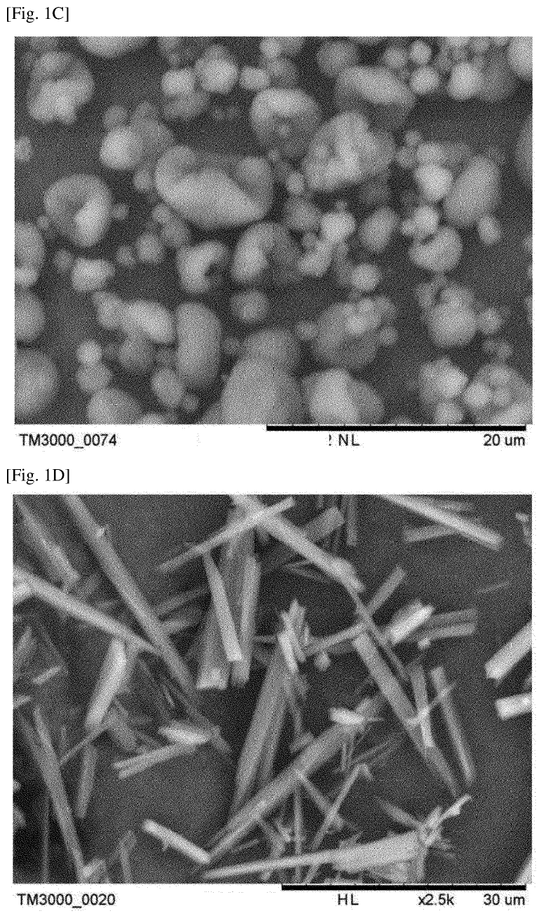

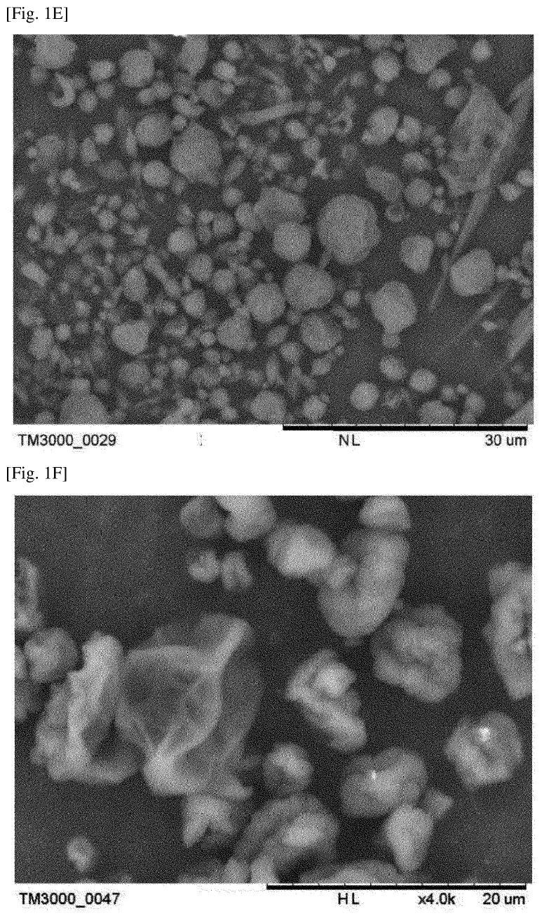

Scanning electron microscopy was carried out (TM3000 SEM, Hitachi, Japan) at a magnification of 4000-fold (FIGS. 1A-1C, 1F) or 2500-fold (FIGS. 1D and 1E) for the Examples and Comparative Examples described above. Needle-shaped crystals of Compound A monohydrate were very prominent in Comparative Example 1 (FIG. 1D), a crystalline control and are also observable among round, aggregated particles in Comparative Example 2 (FIG. 1E). Such crystalline needles are absent in the solid dispersions of Examples 1-3 (FIGS. 1A-1C), characterized by the presence of smooth, round aggregated particles. Comparative Example 3, an excess polymeric carrier control, albeit free of crystalline needles, is characterized by rough aggregates with inhomogeneity in particle size.

### 2. Saturation Solubility

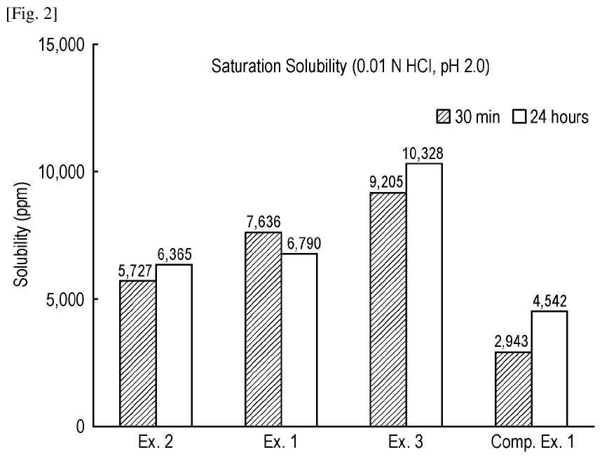

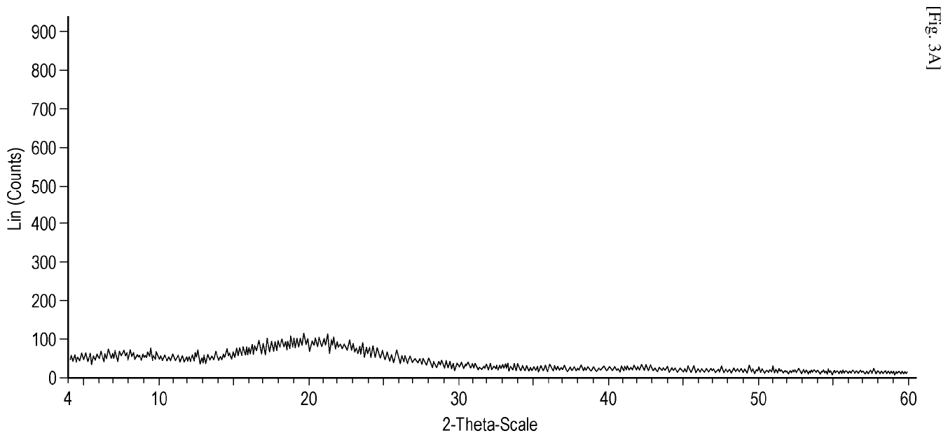

Saturation solubility was evaluated for formulations of Examples 1-3 and Comparative Example 1. Briefly, the spray-dried material was measured and pre-treated in 0.01 N HCl (pH 2.0) using a multitube vortexer with stirring at 1,000 rpm, the amount being set for a theoretical Compound A concentration of 50,000 μg/mL. The stirring was maintained until solubility measurements were taken at either 30 minutes or 24 hours thereafter by letting the test solution to stand for an hour. The test solution was then centrifuged at 3,000 rpm for 10 minutes and the supernatant was filtered with a 0.45 m PVDF filter to pre-treat for the ensuing HPLC assay. As shown in FIG. 2, the amorphous solid dispersions of Examples 1-3 all exhibited superior solubility over the crystalline control of Comparative Example 1. The solubility correlated with the polymeric carrier content.

### 3. Crystallinity

Crystallinity of the Compound A was evaluated for formulations of Example 1 and Comparative Example 1. X-ray diffractometry was carried out with CuKα radiation (40 kV, 40 mA) in the 20 range of 4.0-450 at a scan speed of 3°/min using Bruker D8 Advance diffractometer. Differential scanning calorimetry (DSC) was carried out at a heating rate of 10°/min with nitrogen in the temperature range of 25-250 °C using Scinco (Korea)N-650 calorimeter.

Compound A in the spray-dried solid dispersion of Example 1 was amorphous as evidenced by lack of characteristic peaks in the diffractogram (FIG. 3A) and absence of any thermal peak in the DSC curve (FIG. 3B). This contrasted with the Compound A of Comparative Example 1 for which sharp XRD peaks (FIG. 3C) as well as well-defined endothermic peak around 160 °C (FIG. 3D) were observable, pointing to high crystallinity.

### Experimental Example 2: Analysis of Tablets Prepared from Amorphous Solid Dispersions of Compound A

In this example, tablets of Compound A based on an amorphous solid dispersion of the present invention were prepared and evaluated for amorphous stability.

Tablets containing 100 mg Compound A monohydrate were formulated as summarized in Table 2. A spray-dried amorphous solid dispersion of Compound A was prepared as described for Experimental Example 1 above, except that the dispersion further comprised a glidant/adsorbent at a level of 1.03% by weight. This dispersion was then first mixed with intragranular excipients using Turbular® mixer (WAB, Switzerland) in 300 rotations. It was then compacted using a hydraulic press at 4 MPa and a roller speed of 4.0 rpm and fed into a screw feeder at a speed of 15 rpm. Then it was granulated with an oscillator at 100 rpm and screened with a mesh size of 1.0 mm. These granules were final mixed with an extragranular lubricant using Turbular® mixer in 60 rotations. The granules were then tableted using Autotab® 200 tableting machine (Ichihashi Seiki Co., Japan) at a target hardness of 12-14 kp and coated with Opadry (Colorcon, USA) using SFC-30F (Sejong Pharmatech, Korea) coater with an intake of 65 °C and exhaust of 45 °C and panning at 4 rpm. The coating pump rate was 2 rpm and the product temperature was at 40-50 °C.

TABLE 2
Compositions of the tablets

| | | | Example | Example | Example | Comparative |
|---|---|---|---|---|---|---|
| | | | A (mg) | B (mg) | C (mg) | Example A (mg) |
| Intra- | Spray- | Compound A | 100 | 100 | 100 | 100 |
| granular | dried | monohydrate | | | | |
| | dis- | HPMC 2910 | 25.0 | 25.0 | 25.0 | 25.0 |
| | persion | Colloidal | 1.3 | 1.3 | 1.3 | 1.3 |
| | | Silica | | | | |
| | Mannitol | | 140.0 | 127.0 | 102.0 | 152.0 |
| | Microcrystalline | | 57.5 | 53.0 | 43.0 | 63.0 |
| | cellulose | | | | | |
| | Crospovidone | | 17.5 | 35.0 | 70.0 | 0.0 |
| | Colloidal silica | | 3.5 | 3.5 | 3.5 | 3.5 |

| | | | | | |
|---|---|---|---|---|---|
| Extra- | Magnesium stearate | 5.2 | 5.2 | 5.2 | 5.2 |
| granular | | | | | |
| Coating | Opadry | 10.5 | 10.5 | 10.5 | 10.5 |

| | | | | |
|---|---|---|---|---|
| Total weight | 360.5 | 360.5 | 360.5 | 360.5 |

The stability of solid amorphous form in three different storage conditions was evaluated (at the 20th day) by means of a representative tablet of the present invention. Example C tablet described above were chosen for testing. The three different storage conditions are summarized in Table 3.

TABLE 3
Storage Stability Test Conditions

| | Temperature | Relative Humidity | Storage |
|---|---|---|---|
| | (°C) | (%) | Period |
| High Temperature | 60 ± 2 | — | 20 days |
| Condition | | | |
| Accelerated | 40 ± 2 | 75 ± 5 | |
| Condition | | | |
| High Humidity | 25 ± 2 | 90 ± 5 | |
| Condition | | | |

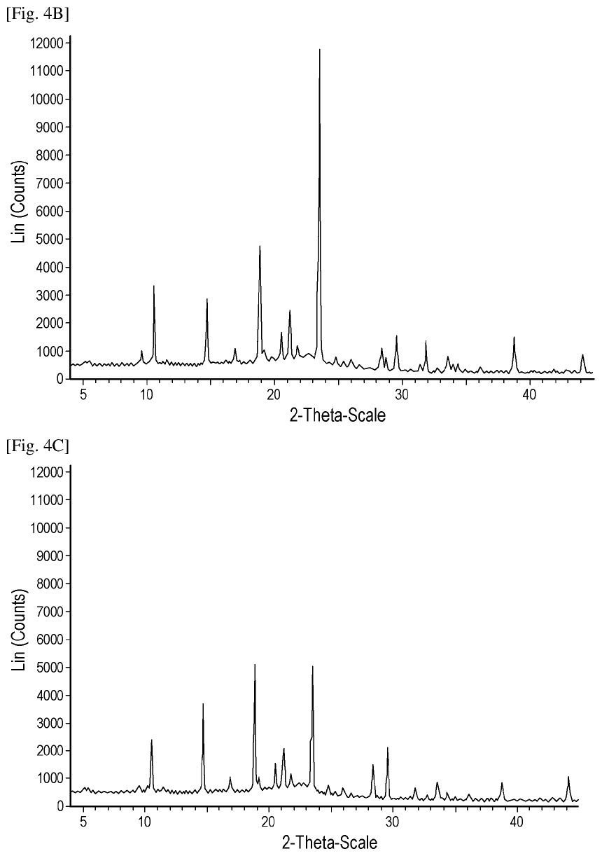

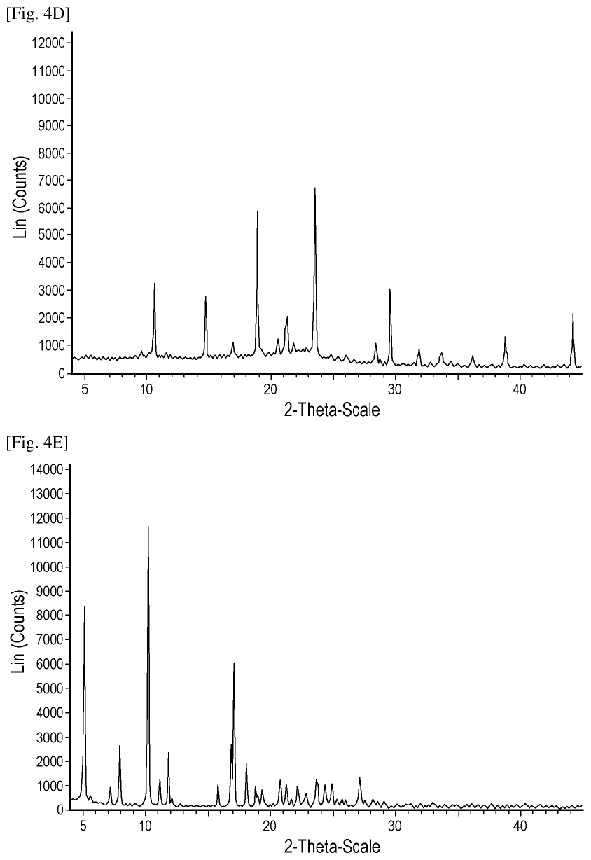

FIG. 4A is the X-ray diffractogram for Example C tablet at the initial time point (note the peaks resulting from other ingredients such as excipients), and FIG. 4E is the diffractogram for the Comparative Example 1 (i.e., crystalline Compound A monohydrate that has not been subject to spray-drying) at the initial time point. The apparent lack of matching peaks in FIG. 4A with those in FIG. 4E is in agreement with the mismatch in polymorphic states of Compound A, (i.e., an amorphous state and a crystalline state) for the two cases. Such absence of peaks at 20 positions corresponding to crystalline peaks was similarly observed in the storage test samples as shown in FIGS. 4B (high temperature), 4C (accelerated) and 4D (high humidity). Thus, the tablet of the present invention was free of polymorphic changes occurring at the storage conditions tested.

### Experimental Example 3: Saturation Solubilities of Different Solid Formulations of Compound A

In this example, saturation solubilities of different solid state formulations comprising the inventive Compound A amorphous solid dispersion were determined. Solubilities determined for a Compound A solid dispersion with glidant/adsorbent (Example 1) and solid dosage formulations of a tablet (Example C) and a capsule (Example D) that incorporate this solid dispersion were compared against a no-dispersion control of previously described Comparative Example 1. The capsule of Example D was prepared by filling a No. 2 capsule with the solid dispersion of Example 1, except that a lubricant was mixed into the dispersion before filling the capsule. The compositions of the solid formulations prepared are summarized in Table 4.

TABLE 4
Compositions of the formulations

| | | | Comp. Ex. | Example 1 | Example C | Example D |
|---|---|---|---|---|---|---|
| | | | 1 (mg) | (mg) | Tablet (mg) | Capsule (mg) |
| Spray- | | Compound A | 100 | 100 | 100 | 100 |
| dried | | monohydrate | | | | |
| dispersion | | HPMC 2910 | — | 25.0 | 25.0 | 25.0 |
| | | Colloidal Silica | — | — | 1.3 | 1.3 |
| Tablet | Intra- | Mannitol | — | — | 102.0 | — |
| | granular | Lactose hydrate | — | — | — | — |
| | | Microcrystalline | — | — | 43.0 | — |
| | | cellulose | | | | |
| | | Crospovidone | — | — | 70.0 | — |
| | | Colloidal silica | — | — | 3.5 | — |
| | Extra- | Magnesium | — | — | 5.2 | — |
| | granular | stearate | | | | |

| | | | | | |
|---|---|---|---|---|---|
| Capsule | Magnesium | — | — | — | 3.5 |
| | stearate | | | | |
| | No. 2 Capsule | — | — | — | 60.0 |
| Coating | Opadry | — | — | 10.5 | — |

| | | | | |
|---|---|---|---|---|
| Total weight | 100 | 125.0 | 360.5 | 189.8 |

Pre-treatment for the saturation solubility measurement was carried as follows. The test solutions were prepared at a target Compound A concentration of 2,000 μg/mL by placing the formulations prepared according to Table 4 in an acetic acid buffer (pH 5.5, prepared according to USP) at ambient temperature, followed by stirring for 30 minutes. The buffer solution was then centrifuged and the supernatant was collected and analyzed with high performance liquid chromatography (HPLC).

TABLE 5
Solubilities of the formulations.

| | Comp. Ex. 1 | Example 1 | Example C | Example D |
|---|---|---|---|---|
| characterization | Non-dispersed, | Spray-dried | Spray-dried | Spray-dried |
| | Crystalline | solid dispersion | solid dispersion | solid dispersion |
| | | | tablet | capsule |
| solubility | No Compound | 1956 | 1526 | 1907 |
| (μg/mL) | A detected | | | |

The results show that solid oral dosage forms employing the inventive Compound A solid dispersion are capable of attaining high solubility, for example, a capsule formulation was able to reach the solubility level of the solid dispersion itself.

### Experimental Example 4: Pharmacokinetic Analysis of Solid Formulations Prepared from Amorphous Solid Dispersions of Compound A

In this example, in vivo experiments were conducted to evaluate the benefit of the solid dispersion formulation over the conventional formulation. Pharmacokinetic properties were determined and compared between tablets prepared with or without the amorphous solid dispersion of the present invention.

A tablet (Example C) or capsule (Example D) was formulated from 100 mg Compound A monohydrate as summarized in Table 6 with spray-drying. For the non-dispersed tablet of Comparative Example B, a simple mixing of excipients was carried out with crystalline Compound A monohydrate without spray-drying, followed by an addition of a lubricant and direct compacting and coating.

TABLE 6
Compositions of the formulations

| | | | | | Comparative |
|---|---|---|---|---|---|
| | | | Example C | Example D | Example B |
| | | | Tablet (mg) | Capsule (mg) | (mg) |
| Spray- | | Compound A | 100 | 100 | 100 |
| dried | | monohydrate | | | |
| dispersion | | HPMC 2910 | 25.0 | 25.0 | — |
| | | Colloidal Silica | 1.3 | 1.3 | — |
| Tablet | Intra- | Mannitol | 102.0 | — | — |
| | granular | Lactose hydrate | — | — | 58.0 |
| | | Microcrystalline | 43.0 | — | 30.0 |
| | | cellulose | | | |
| | | Crospovidone | 70.0 | — | 6.0 |
| | | Colloidal silica | 3.5 | — | 2.0 |
| | Extra- | Magnesium stearate | 5.2 | — | 4.0 |
| | granular | | | | |

| Capsule | Magnesium stearate | — | 3.5 | — |
|---|---|---|---|---|
| | No. 2 Capsule | — | 60.0 | — |
| Coating | Opadry | 10.5 | — | 6.0 |

| | | | |
|---|---|---|---|
| Total weight | 360.5 | 189.8 | 206.0 |

Each of the resultant formulation was fed to fasted male beagle dogs (n=4) in a single oral administration of 100 mg Compound A to the test groups in parallel and the pharmacokinetic parameters were determined by taking blood samples at fixed time points. The results are summarized in Table 7.

TABLE 7
PK parameters of the tablets.

| | Example C tablet | Example D capsule | Comp. Ex. B tablet |
|---|---|---|---|
| AUCₗₐₛₜ (ng · hr/mL) | 5604.6 ± 3014.6 | 6060.9 ± 1803.9 | 2123.7 ± 1997.6 |
| Cₘₐₓ (ng/mL) | 508.3 ± 334.0 | 477.8 ± 105.2 | 168.4 ± 162.0 |
| Tₘₐₓ (hours) | 4.0 ± 0.0 | 6.0 ± 2.3 | 4.0 ± 0.0 |

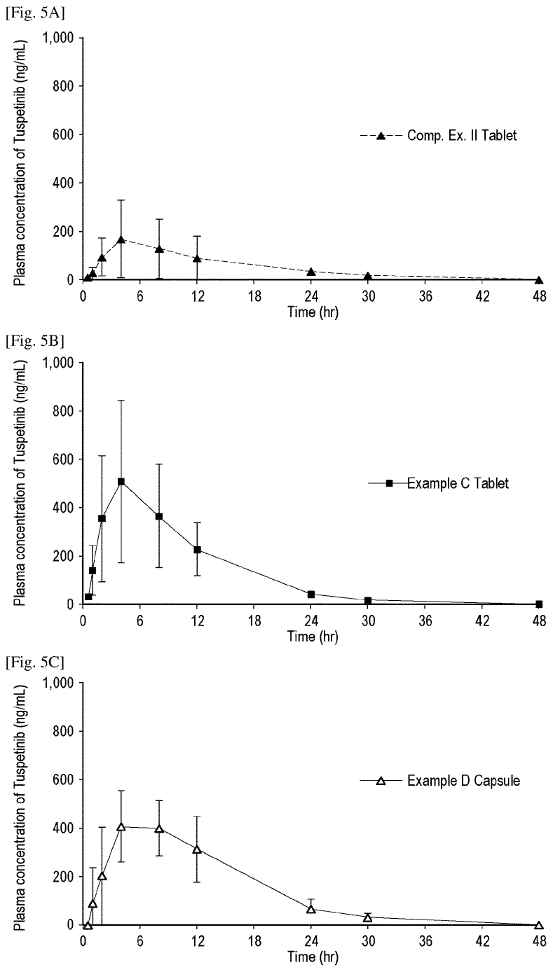

FIGS. 5A-5C show the mean plasma concentration-time profiles of the solid formulations. Example C, a tablet according to the present invention, with the application of solid dispersion, was capable of attaining higher solubility, leading to notable improvement in drug exposure and absorption. Example C tablet reached the maximal plasma concentration (Cₘₐₓ) of Compound A in the interval between 3 to 10 hours with the time to reach Cₘₐₓ occurring after 3 hours in beagle dogs. Example C tablet exhibited about 3-fold increase in drug exposure (Cₘₐₓ) and about 2.6-fold area under the curve (AUC) over the tablet of Comparative Example B. Example D, a capsule according to the present invention, with the application of solid dispersion, was also capable of attaining higher solubility, drug exposure and absorption. Example D capsule exhibited about 2.8-fold increase in drug exposure (Cₘₐₓ) and about 2.9-fold area under the curve (AUC) over the tablet of Comparative Example B.

### Formulation Example 1: Spray-Dried Amorphous Solid Dispersion Example of Compound A

Spray-dry dispersion of Compound A can be made with hydroxypropyl methyl cellulose acetate succinate (HPMCAS) as the polymeric carrier. Table 8 provides an exemplar composition for this spray-dry dispersion based on HPMCAS. The same procedure described in Experimental Example 1 can be used for preparation except for HPMCAS (preferably with suitably low pH solubility threshold, e.g., AQOAT® LF Hypromellose Acetate Succubate, Shin-Etsu, Japan) replacing the HPMC as well as the adjustment in the amounts of water and ethanol used as indicated in Table 8 in consideration of the change in solubility.

TABLE 8
Composition of the HPMCAS formulation

| | Compound A | HPMCAS | | | Final |
|---|---|---|---|---|---|
| | (mg) | (mg) | Water (mg) | Ethanol (mg) | Weight (mg) |
| Formulation | 100.0 | 25.0 | 400 | 4600 | 125.0 |

### Formulation Example 2: Non-Spray-Dried Solid Dispersion Examples

### 1. Amorphous Solid Dispersion of Compound a by Reduced Pressure Drying

A Compound A working suspension is prepared by mixing a Compound A ethanol solution with a HPMC aqueous solution in which silica is suspended. This working suspension can be dried with a vacuum drier at reduced pressure for 24 hours. The dried mixture can then be ground by milling in a mixer and passed through a 30-mesh circular sieve to yield the amorphous solid dispersion of Compound A.

### 2. Preparation of Amorphous Solid Dispersion of Compound a Tablet by Fluid Bed Granulation

Table 9 provides an exemplar composition for amorphous solid dispersion tablet of Compound A based on HPMC by fluid bed granulation.

TABLE 9
Composition for fluid bed granulation

| | | | Weight (mg) |
|---|---|---|---|
| Fluid | Fluid Bed | Compound A | 100 |
| Bed | Granulation | monohydrate | |
| Granule | Liquid | HPMC 2910 | 25.0 |
| | | Colloidal | 1.3 |
| | | Silica | |
| | | Deionized | (3,200) |
| | | Water | |
| | | Ethanol | (800) |
| | Mannitol | | 102.0 |
| | Microcrystalline cellulose | | 43.0 |
| Post-mix | Crospovidone | | 70.0 |
| | Colloidal silica | | 3.5 |
| Final- | Magnesium stearate | | 5.2 |
| mix | | | |

| | | |
|---|---|---|
| Coating | Opadry | 10.5 |

| | |
|---|---|
| Total weight | 360.5 |

In summary, the amorphous solid dispersion formulations of Compound A comprising the spray-dried form proved to be advantageous over a conventional tablet of the same dose non-reliant on such solid dispersion technology.

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A solid dispersion comprising Compound A: 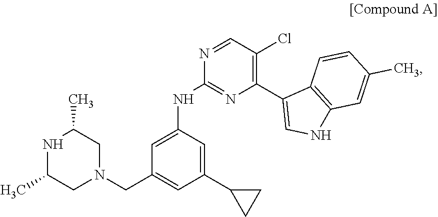 [SMILES: Cc1ccc2c(-c3nc(Nc4cc(CN5C[C@@H](C)N[C@@H](C)C5)cc(C5CC5)c4)ncc3Cl)c[nH]c2c1]
or a pharmaceutically acceptable salt, solvate, stereoisomer or prodrug thereof.

2. The solid dispersion of claim 1, wherein the solid dispersion comprises a polymeric carrier.

3. The solid dispersion of claim 2, wherein the polymeric carrier is in an amount of from about 10 parts to about 55 parts by weight based on 100 parts by weight of Compound A; and wherein Compound A is substantially amorphous and present in a polymer matrix of the polymeric carrier.

4. The solid dispersion of claim 1 or claim 2, wherein the polymeric carrier is in an amount from about 10 parts to about 50 parts by weight based on 100 parts by weight of Compound A.

5. The solid dispersion of any one of claims 2-4, wherein the polymeric carrier is one or more selected from the group consisting of methyl cellulose, ethyl cellulose, hydroxyethyl cellulose, hydroxypropyl cellulose, carboxymethyl cellulose, cellulose acetate, cellulose acetate phthalate, hydroxypropylmethyl cellulose (HPMC), hydroxypropylmethyl cellulose phthalate (HPMCP), hydroxypropylmethyl cellulose acetate succinate (HPMCAS), polyvinyl pyrrolidine (PVP), poly vinyl pyrrolidine-vinyl acetate copolymer (PVP-VA), crospovidone, polyethylene glycol (PEG), methyl cellulose (MC), methylacrylic acid methyl methacrylate copolymer, poly(acrylic acid), cellulose acetate phthalate (CAP), chitosan, polyethylene glycol-vinyl acetate copolymer (PEG-PVA), graft copolymer of ethylene glycol-vinyl caprolactam-vinyl acetate, polyvinyl acetate phthalate (PVAP), polyoxyethylene-polyoxypropylene block copolymer and D-α-tocopherol polyethylene glycol succinate (TPGS).

6. The solid dispersion of claim 2, wherein the polymeric carrier is a hydrophilic carrier.

7. The solid dispersion of any one of claims 2-6, wherein the polymeric carrier is one or more cellulose derivative selected from the group consisting of hydroxypropyl methyl cellulose (HPMC), hydroxypropyl methyl cellulose phthalate (HPMCP), hydroxypropyl methyl cellulose acetate succinate (HPMCAS), methyl cellulose, ethyl cellulose, hydroxyethyl cellulose, hydroxypropyl cellulose, carboxymethyl cellulose, cellulose acetate and cellulose acetate phthalate.

8. The solid dispersion of claim 1, wherein the solid dispersion is formed by a solvent evaporation method, hot-melt extrusion or spray drying dispersion.

9. The solid dispersion of claim 8, wherein the solvent evaporation method is spray drying or fluid bed granulation.

10. The solid dispersion of claim 1, wherein the solid dispersion further comprises a glidant/adsorbent.

11. A pharmaceutical composition comprising a therapeutically effective amount of Compound A having the structure: 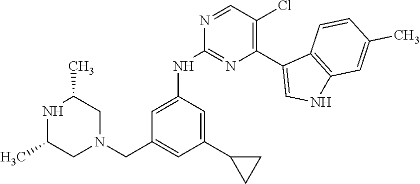
or a pharmaceutically acceptable salt, solvate, stereoisomer or prodrug thereof, wherein the composition is a solid dispersion.

12. The pharmaceutical composition of claim 11, wherein the solid dispersion comprises a polymeric carrier.

13. The pharmaceutical composition of claim 11 or 12, comprising
a) from about 20% w/w to about 50% w/w of Compound A solid dispersion;
b) from about 25% w/w to about 70% w/w of filler/binder;
c) from about 4.0% w/w to about 20% w/w of disintegrant;
d) from about 0.25% w/w to about 5.0% w/w of glidant/adsorbent; and
e) from about 0.5% w/w to about 2.5% w/w of lubricant;
wherein the Compound A solid dispersion comprises substantially amorphous Compound A dispersed in a polymer matrix of a hydrophilic polymeric carrier in an amount of from about 64.5% w/w to about 91% w/w Compound A based on the combined weight of the Compound A and the hydrophilic polymeric carrier.

14. The pharmaceutical composition of claim 13, wherein the pharmaceutical composition is a solid oral dosage form selected from the group consisting of tablets, powders, granules, caplets and pills.

15. The pharmaceutical composition of claim 14, wherein the solid oral dosage form is a tablet.

16. The pharmaceutical composition of any one of claims 11-15, wherein the hydrophilic polymeric carrier is hydroxypropyl methyl cellulose.

17. The pharmaceutical composition of any one of claims 13-16, wherein the filler/binder is one or more selected from the group consisting of starch, saccharides, disaccharides, sucrose, lactose, lactose monohydrate, polysaccharides, cellulose, cellulose ethers, hydroxypropylcellulose, sugar alcohols, xylitol, sorbitol, maltitol, microcrystalline cellulose, calcium or sodium carbonate, dicalcium phosphate, compressible sugars, dibasic calcium phosphate dehydrate, mannitol, tribasic calcium phosphate, and any combination thereof.

18. The pharmaceutical composition of any one of claims 13-17, wherein the disintegrant is one or more selected from the group consisting of starch, sodium starch glycolate, croscarmellose sodium, crospovidone, microcrystalline cellulose, modified corn starch, sodium carboxymethyl starch, povidone, pregelatinized starch, alginic acid and any combination thereof.

19. The pharmaceutical composition of any one of claims 13-18, wherein the glidant/adsorbent is one or more selected from the group consisting of colloidal silicon dioxide, talc, fumed silica, starch, starch derivatives, bentonite and any combination thereof.

20. The pharmaceutical composition of any one of claims 13-19, wherein the lubricant is one or more selected from the group consisting of magnesium stearate, stearic acid, silica, fats, calcium stearate, polyethylene glycol, sodium stearyl fumarate, or talc, lauric acid, oleic acid, C8/C10 fatty acid and any combination thereof.

21. The pharmaceutical composition of any one of claims 11-20, wherein the Compound A solid dispersion is formed by spray-drying.

22. The pharmaceutical composition of any one of claims 11-21, wherein the tablet provides about 2-fold to about 3-fold increase in drug exposure over a reference tablet comprising an identical amount of Compound A but not comprising an amorphous solid dispersion of Compound A.

23. The pharmaceutical composition of claim 22, wherein the drug exposure comparison is based on beagle dogs.

24. The pharmaceutical composition of claim 15, wherein the tablet provides about 2-fold to about 3-fold increase in area under the curve (AUC) over a reference tablet comprising an identical amount of Compound A but not comprising an amorphous solid dispersion of Compound A.

25. The pharmaceutical composition of claim 24, wherein the area under the curve comparison is based on beagle dogs.

26. A pharmaceutical composition for capsule comprising
a) from about 50% w/w to about 97.5% w/w of Compound A solid dispersion;
b) from about 0% w/w to about 48% w/w of filler/binder;
c) from about 0% w/w to about 20% w/w of disintegrant;
d) from about 0% w/w to about 4.0% w/w of glidant/adsorbent; and
e) from about 0.2% w/w to about 4.0% w/w of lubricant;
wherein Compound A is 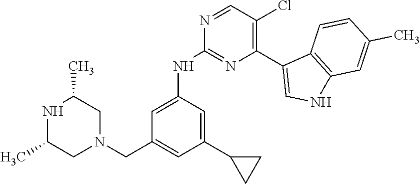
[Compound A], or a pharmaceutically acceptable salt, solvate, stereoisomer or prodrug thereof, and wherein the Compound A solid dispersion comprises substantially amorphous Compound A dispersed in a polymer matrix of a hydrophilic polymeric carrier in an amount of from about 64.5% w/w to about 91% w/w Compound A based on the combined weight of the Compound A and the hydrophilic polymeric carrier.

27. A solid oral formulation comprising from about 20% to about 50% w/w of a Compound A solid dispersion,
wherein the oral formulation provides about 2-fold to about 3-fold increase in drug exposure over a reference tablet comprising an identical amount of Compound A but not comprising an amorphous solid dispersion of Compound A,
wherein the Compound A solid dispersion comprises substantially amorphous Compound A dispersed in a polymer matrix of hydroxypropyl methyl cellulose in an amount of from about 64.5% w/w to about 91% w/w Compound A based on the combined weight of the Compound A and hydroxypropyl methyl cellulose.

28. A solid oral formulation comprising from about 20% w/w to about 50% w/w of Compound A solid dispersion,
wherein the oral formulation provides about 2-fold to about 3-fold increase in area under the curve over a reference tablet comprising an identical amount of Compound A but not comprising an amorphous solid dispersion of Compound A, and
wherein the Compound A solid dispersion comprises substantially amorphous Compound A dispersed in a polymer matrix of hydroxypropyl methyl cellulose in an amount of from about 64.5% w/w to about 91% w/w Compound A based on the combined weight of the Compound A and hydroxypropyl methyl cellulose.

29. The solid oral formulation of claim 27 or claim 28, wherein the Compound A solid dispersion is formed by spray drying.

30. A method for treating cancer, comprising administering the solid dispersion, pharmaceutical composition or solid oral formulation of any one of claims 1-29, to a subject in need thereof.

31. The method of claim 30, wherein the cancer is a leukemia such as acute myelogenous leukemia (AML), chronic myelogenous leukemia (CML), acute lymphocytic leukemia (ALL), chronic lymphocytic leukemia, CLL), acute promyelocytic leukemia (APL), hairy cell leukemia, or chronic neutrophilic leukemia (CNL).

32. The method of claim 31, wherein the leukemia is AML.

33. The method of any one of claims 30-32, wherein the cancer or leukemia comprises cancer cells having a mutation in the FLT3 gene.

34. The method of claim 33, wherein the FLT3 gene has an internal tandem duplication (ITD) mutation.

35. The method of claim 33 or claim 34, wherein the FLT3 gene has at least one FLT3 mutation selected from F691L, D835Y, D835F, D835I, D835H, D835V and D835A.

36. An amorphous form of Compound A: 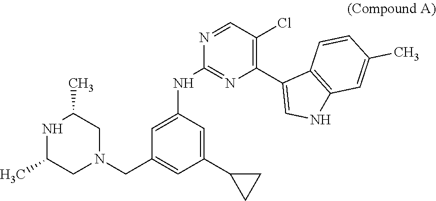
or a pharmaceutically acceptable salt, solvate, or solvate salt thereof; wherein the amorphous form exhibits an X-ray powder diffraction (XRPD) pattern substantially similar to the pattern in FIG. 3A.

37. An amorphous form of Compound A: 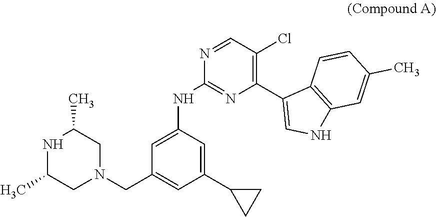
or a pharmaceutically acceptable salt, solvate, or solvate salt thereof; wherein the amorphous form exhibits an X-ray powder diffraction (XRPD) pattern as in FIG. 3A.

38. The amorphous form of Compound A, or a pharmaceutically acceptable salt, solvate, or solvate salt thereof; which exhibits a differential scanning calorimetry (DSC) curve substantially similar to FIG. 3B.

39. The amorphous form of any one of claims 36-38 having a purity in the range of about 80% to about 99%.

40. The amorphous form of any one of claims 36-38 having a purity of about 95% or higher.

41. The amorphous form of any one of claims 36-38 having a purity of about 99% or higher.

42. The amorphous form of any one of claims 36-41 comprising less than 10% of crystalline form of Compound A or a pharmaceutically acceptable salt, solvate, stereoisomer or prodrug thereof.

43. The amorphous form of any one of claims 36-41 comprising less than 5% of crystalline form of Compound A or a pharmaceutically acceptable salt, solvate, stereoisomer or prodrug thereof.

44. A means for inhibiting Myeloid kinome, wherein the myeloid kinome means FMS-like tyrosine kinase 3(FLT3), spleen tyrosine kinase(SYK), Janus kinase(JAK) and/or mutant c-KIT.

45. A means for binding to or inhibiting FMS-like tyrosine kinase 3 (FLT3).

46. The means of claim 44 or 45, wherein the means are in an amorphous form.

47. The means of any one of claims 44-46, wherein the means are in a free base form.

48. The means of any one of claims 44-47, wherein the means are in a monohydrate form.
